(19) United States
(12) Patent Application Publication
(10) Pub. No.: US 2020/0207806 A1
(43) Pub. Date: Jul. 2, 2020

(54) MANUFACTURE OF ENDOTOXIN-FREE HEMOGLOBIN-BASED DRUG SUBSTANCE AND METHOD FOR ENDOTOXIN-FREE PROTEIN PURIFICATION

(71) Applicant: Medical Technology Associates II, Inc., Durham, NH (US)

(72) Inventor: Carl W. Rausch, Lawrence, MA (US)

(21) Appl. No.: 16/643,269

(22) PCT Filed: Sep. 12, 2018
(86) PCT No.: PCT/US18/50623

§ 371 Date: Feb. 28, 2020

Related U.S. Application Data
(60) Provisional application No. 62/557,324, filed on Sep. 12, 2017.

(51) Int. Cl. C07K 1/34; C07K 1/16; C07K 14/805

(52) U.S. Cl. CPC C07K 1/34; B01D 69/08; C07K 14/805; C07K 1/16

(57) ABSTRACT

The present invention relates to the surprising discovery that previous hemoglobin-based drug purification methodologies do not remove sufficient endotoxins exposures at the various steps which may complex with the hemoglobin protein. These complexed endotoxins can result in serious health complications (e.g. development of cardiac lesions for one). Additionally, varied endotoxin types and concentration contributes to batch-to-batch variability during hemoglobin-based drug manufacture. Endotoxins are not as much of an issue for peptides as compared to larger protein complexes. Accordingly, the instant disclosure is directed to a purification process using single use systems in many process steps including high performance chromatography systems thereby removing endotoxins while keeping processing costs low.

### BACKGROUND OF THE INVENTION

The development of hemoglobin-based drugs, such as hemoglobin-based oxygen carriers, has been based on oxygen delivery for use in medical therapies such as transfusions and the production of blood products. Hemoglobin-based drugs were proposed to be used to prevent or treat hypoxia resulting from red blood cell loss, “blood loss” (e.g. from acute hemorrhage or during surgical operations), from anemia (insufficient oxygen carriage via the circulation) (e.g., pernicious anemia or sickle cell anemia and acute hemodilution), or from shock (e.g., volume deficiency shock, septic shock or hemorrhagic shock).

Existing hemoglobin-based drugs and oxygen carriers include perfluorochemicals, synthesized hemoglobin analogues, liposome-encapsulated hemoglobin, chemically-modified hemoglobin, and hemoglobin-based oxygen carriers in which the hemoglobin molecules are crosslinked. Preparation of hemoglobin-based drugs includes several purification steps to remove agents and cellular components that cause severe immune responses. Among the components that must be removed from hemoglobin-containing fluids (e.g. collected blood) is fibrinogen, which is a soluble protein that is converted into fibrin by the action of thrombin during clotting and the cellular surface materials and immunoglobulins that can create and inconsistency of hemoglobin agents to be specific. Current techniques for processing blood often include addition of chemical agents, such as sodium citrate, to prevent coagulation. However, additional techniques which might, for example, reduce the expense of processing, without diminishing other qualities, such as ultimate product purity, are sought. Unfortunately, existing methods of producing hemoglobin solutions from bovine blood utilize drug purification methodologies that most of the time do not remove completely such elements as the lipid layers of the cells and more specifically the lipopolysaccharides (endotoxins) which can complex with the hemoglobin protein at any stage of handling given exposure to bacteria endotoxin materials, as such, there is a pressing need to provide a method of hemoglobin-based drug purification and handling that are more cost effective, has increased product purity, and has better batch reproducibility compared to previous techniques. All of this to set forth reasonable and reproducible processing environments.

### SUMMARY OF THE INVENTION

The present invention is based upon the discovery that previous hemoglobin-based drug purification methodologies do not remove many components that are considered foreign and that create variances in time of processing (protein denaturation) and more specifically, endotoxins which complex with the hemoglobin protein. These specific complexed endotoxins can result in serious health complications (e.g. development of cardiac lesions). Additionally, varied endotoxin types and concentration contributes to batch-to-batch variability during hemoglobin-based drug manufacture. Endotoxins are not as much of an issue for peptides as compared to larger protein complexes for they can be ultrafiltered in many cases.

Accordingly, described herein are methods of purifying hemoglobin-based oxygen carriers such that endotoxins and other such materials are removed and processing costs remain low.

### DETAILED DESCRIPTION OF THE INVENTION

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

By “agent” is meant any small protein based or other compound, antibody, nucleic acid molecule, or polypeptide, or fragments thereof.

By “alteration” is meant a change (increase or decrease) in the molecular weigh distribution of a stabilization technique or reaction as detected by standard art-known methods such as those described herein. As used herein, an alteration includes at least a 5% change in crosslinked levels, e.g., at least a 5% to 95, or 100% change in cross-linked molecular stabilization levels. For example, an alteration includes at least a 5%-10% change in protein stabilization, preferably a 25% change, more preferably a 80% change, and most preferably a 590% or greater change in stabile molecular size.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reacting substance to form a covalent bond (e.g, glutaraldehyde). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

By the terms “effective amount” and “therapeutically effective amount” of a formulation or formulation component is meant a sufficient amount of the formulation or component, alone or in a combination, to provide the desired effect. For example, by “an effective amount”is meant an amount of a compound, alone or in a combination, required to ameliorate the symptoms of an anemic and or iron deficient state, e.g., hypoxia, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a protein molecule. This portion contains, preferably, at least the heme iron portion of the molecule or original protein construct of hemoglobin. For example, a fragment may contain 1,2 or 4 side chains of the alpha nd bets fragments of the native hemoglobin molecule. However, the invention also comprises the protein fragments, so long as they exhibit the desired biological activity from the full length globular protein structure For example, illustrative poly-amino acid segments with total weights of about 16,000 Kd, about 32,000 kd, in size (including all intermediate weights) are included in many implementations of this invention. Similarly, a protein fragment of almost any length is employed if it is the iron carrier (heme group).

The terms “isolated,” “purified, ” or “biologically pure” refer to material that is free to varying degrees from components which normally accompany it as found in its native environment. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, stabilized protein of a fragment to a polymer in this invention, it is purified if it is substantially free of cellular material, viral material, or culture medium when produced by recombinant DNA techniques, or chemical precursors or other chemicals when chemically synthesized, and all other stromal red blood cell or other blood proteins or blood components and cellular debris. Purity, homogeneity and stability are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation, glycosylation, or polymerization different modifications may give rise to different isolated proteins, which can be separately purified.

Similarly, by “substantially pure” is meant a protein or polypeptide that has been separated from the components that naturally accompany it. Typically, the proteins and polypeptides are substantially pure when they are at least 95%, or even 99%, by weight, free from the other proteins and naturally-occurring organic molecules with with they are naturally associated.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide fraction and or protein of the invention may be obtained, for example, by extraction from a natural source, by expression of a recombinant nucleic acid encoding such a material; or by chemically synthesizing the protein. Purity can be measured by any appropriate method, for example, column chromatography, polyacrylamide gel electrophoresis, or by HPLC analysis.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By “marker” is meant any protein or polynucleotide having an alteration in expression level or activity that is associated with a disease or disorder, e.g., neoplasia.

By “modulate” is meant alter (increase or decrease). Such alterations are detected by standard art-known methods such as those described herein.

The term, “normal amount” refers to a normal amount of a complex in an individual known not to be diagnosed with cancer or various metabolic and physiologic disease states. The amount of the molecule can be measured in a test sample and compared to the “normal control level,” utilizing techniques such as reference limits, discrimination limits, or risk defining thresholds to define cutoff points and abnormal values (e.g., for neoplasia, hypoxia, ischemia). The “normal control level” means the level of one or more proteins (or nucleic acids) or combined protein indices (or combined nucleic acid indices) typically found in a subject known not to be suffering from cancer or the physiologic oxygen deficient status. Such normal control levels and cutoff points may vary based on whether a molecule is used alone or in a formula combining other proteins into an index. Alternatively, the normal control level can be a database of protein patterns from previously tested subjects who did not convert to cancer over a clinically relevant time horizon. It can also be a condition of reduced oxygen tension as measure in mmHg as characterized as hypoxic or ischemic. In another aspect, the normal control level can be a level relative to a regular cellular function and the level of oxygenation.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed and assessed in that the control does not suffer from the disease in question or is not at risk for the disease or reflects signs and symptoms of oxygen depravation.

Relative to a control level, the level that is determined may be an increased level. As used herein, the term “increased” with respect to level (e.g., expression level, biological activity level, etc.) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Protein molecules useful in the methods of the invention include any nucleic acid molecule that encodes a polypeptide of heme iron composition of the invention or a fragment thereof. Such protein stabilized molecules need not be 100% identical with an endogenous nucleic acid sequence, but will typically exhibit substantial identity, e.g., at least 80%, at least 85%, at least 90%, at least 95%, or at least 99% identity.

For most applications, washing steps that follow hybridization will also vary in stringency. Wash/ and Mix conditions stringency controlled can be defined by buffer concentrations, glutaraldehyde reactions conditions of dispersion and by temperature. As above, controlled stringency can be increased by decreasing salt concentration or by increasing temperature. Additional variations on these conditions will be readily apparent to those skilled in the art. Hybridization/conjugation techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; gelatin; excipients; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from neoplasias or circulating exosomes. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The strip may be a nucleic acid-probe (or protein) coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody or nucleic acid molecule). Thus, the support configuration may be spherical, as in a bead, or cylindrical, as in the inside surface of a test tube, or the external surface of a rod. Alternatively, the surface may be flat such as a sheet, or test strip, etc. For example, the supports include polystyrene beads. Those skilled in the art will know many other suitable carriers for binding antibody or antigen, or will be able to ascertain the same by use of routine experimentation. In other aspects, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains a binding agent alone or together with a binding agent for at least one, two, three or more other molecules.

By “specifically binds” is meant a compound or antibody that recognizes and binds a polypeptide of the invention, but which does not substantially recognize and bind other molecules in a sample, for example, a biological sample, which naturally includes a polypeptide/conjugated purified protein of the invention.

By “substantially identical” is meant a polypeptide/protein or nucleic acid molecule exhibiting at least 80% identity to a reference amino acid sequence (for example, any one of the amino acid sequences described herein) or nucleic acid sequence (for example, any one of the nucleic acid sequences described herein). Preferably, such a sequence is at least 80%, at least 85%, at least 90%, at least 95%, or at least 99% identical at the amino acid level or nucleic acid to the sequence used for comparison.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

In some cases, a composition of the invention is administered orally or systemically. Other modes of administration include topical, intraocular, buccal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration may be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness.

Pharmaceutical compositions may be assembled into kits or pharmaceutical systems for use in adjunctive therapy for cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is an image of a fluid (e.g. blood) from which purified hemoglobin can be obtained.

FIG. 2 is a schematic of a cell washing process step for purification of proteins (e.g. hemoglobin) from a fluid.

FIG. 3 is a schematic of a cell lysis process for purification of protein (e.g. hemoglobin) solution.

FIG. 4 is a schematic of a process for deoxygenation and filtration of a protein (e.g. hemoglobin) solution.

FIG. 5 is a schematic of an anion exchange chromatography purification process for filtration of a protein (e.g. hemoglobin) solution

FIG. 6A-FIG. 6B are schematics of a protein (e.g. hemoglobin) deoxygenation process. FIG. 6A is a schematic of a concentration and deoxygenation system for the first step of protein solution deoxygenation. FIG. 6B is a schematic of a buffer exchange and filtration system for the second step of protein solution deoxygenation.

FIG. 7 is a schematic of a polymerization process for the stabilization of a protein (e.g. hemoglobin).

FIG. 8 is a schematic of a borohydride reduction process.

FIG. 9 is a schematic depicting an alternate embodiment of a cell washing process for purification of proteins (e.g. hemoglobin) from a fluid.

FIG. 10 is a schematic depicting an alternate embodiment of a cell lysis process for purification of protein (e.g. hemoglobin) solution.

FIG. 11 is a schematic depicting an alternate embodiment of a process for deoxygenation and filtration of a protein (e.g. hemoglobin) solution.

FIG. 12 is a schematic depicting an alternate embodiment of an anion exchange chromatography purification process for filtration of a protein (e.g. hemoglobin) solution

FIG. 13A-FIG. 13B are schematics depicting alternate embodiments of a protein (e.g. hemoglobin) deoxygenation process. FIG. 13A is a schematic depicting an alternate embodiment of a concentration and deoxygenation system for the first step of protein solution deoxygenation.

FIG. 13B is a schematic depicting an alternate embodiment of a buffer exchange and filtration system for the second step of protein solution deoxygenation.

FIG. 14 is a schematic depicting an alternate embodiment of a polymerization process for the stabilization of a protein (e.g. hemoglobin).

FIG. 15 is a schematic depicting an alternate embodiment of a borohydride reduction process.

FIG. 16 is a schematic depicting a sterile filtration process for a protein (e.g. hemoglobin) solution.

FIG. 17 is an image of a device for cell recovery or centrate clarification (e.g. CARR Centritech's UniFuge).

FIG. 18 is an image of a separation system (e.g. CARR UniFuge Pilot Centritech Separation System) with features such as single-use disposable module, no CIP or SIP necessary, fully automated, high cell recovery rates, mammalian and insect cell processing potential, integrated trolley, intuitive software, low shear processing, and minimal reduction in viability of recovered cells. Device may be created in state-of-the-art manufacturing facility.

FIG. 19A-FIG. 19B are images of a separation chamber (e.g. UniFuge single use “GR-AC” separation chamber) with features such as glass-reinforced feed and centrate tubes, advanced core with vane accelerator flange, and 0.2″ clearance. Specifications for the device include feed flow range of 0.1-4.0 per minute. FIG. 19A is a perspective view of a separation chamber (e.g. UniFuge single use “GR-AC” separation chamber). FIG. 19B is a top view of a separation chamber (e.g. UniFuge single use “GR-AC” separation chamber).

FIG. 20 is an image of a typical installation of a separation chamber and tubset fully assembled module in a system (e.g. UniFuge system).

FIG. 21 is an image of a separation chamber and tubeset fully assembled (e.g. UniFuge single use “GR-AC” module) with features such as 4-pinch valve configuration, glass-reinforced feedtube and centrate tube, advanced core with vane accelerator flange 0.2″ clearance, includes Meissner filter and tubeset with 24″/18″ C-flex. Feed flow range may be 0.1-4.0 L per minute.

FIG. 22 is a series of images of a tubeset assembly (e.g. UniFuge tubeset assembly) with features such as 4-pinch valve with Meissner filter, 24″ long ⅜″ I.D. C-flex connection tubes. The tubeset assembly uses item a-item u. Item a is a ½″ ID×¾″ OD tubing pharmed 36.00″ OAL that may be part number (no.) P003. Item b is a ½″ WYE connector polypro that may be part no. P006. Item c is a ½″ ID×¾″ OD tubing platinum cured silicone 36.00″ OAL that may be part no. P002. Item d is a ½″ straight connector, polypro that may be part no. P005. Item e is a ½″ ID×¾″ OD tubing37 C-flex 24.00″ OAL that may be part no. P004. Item f is a ½″ tube plug polypro that may be part no. P007. Item g is a large tubing clamp poly that may be part no. P027. Item h is yellow tape that may be part no. P076. Item i is green tape that may be part no. P075. Item j is a ½″ ID×¾″ OD tubing platinum cured silicone 6.00″ OAL that may be part no. P002. Item k is a ½″ pressure sensor polycarbonate that may be part no. P009. Item 1 is a 3/16″ ID× 3/16″ OD tubing platinum cured silicone 18.00″ OAL that may be part no. P015. Item m is a 3/16″ ID Meissner HB 0.2 steridyne filter, CFVMV 0.2-33A1 that may be part no. P016. Item n is a 3/16″ ID× 5/16″ OD tubing platinum cured silicone 4.00″ OAL that may be part no. P0015. Item o is a MIN cable tie used for ¼″- 5/16″ ID tubing that may be part no. P063. Item p is a STD cable tie used for ⅜″ and above ID tubing that may be part no. P062. Item q is blue tape that may be part no. P074. Item r is white tape that may be part no. P080. Item s is a ½″×⅜″ reducer polypro that may be part no. P052. Item t is a ⅜″ ID×⅚″ OD tubing 37 C-flex 18.00″ OAL that may be part no. P050. Item u is a ⅜″ tube plug plypro that may be part no. P053.

FIG. 24 is an image of a Millipore Clarisolve 60HX or like device for blood depth filtration (60 μm and 0.027 m²/0.29 ft²).

FIG. 25 is an image of a Millipore Clarisolve 60HX or like device connected to an assembly for blood depth filtration.

FIG. 26 is a chart depicting an example of protein cross-linking distribution for polymerization step data.

FIG. 27 is a series of graphs depicting protein cross-linking distribution polymerization step data. Various protein peaks at different stages of cross-linking are displayed.

FIG. 28 is an image of polymerization step assembly. Different glutaraldehyde/bHB proportions and types of manifold were tested. Three polymerization reactions were performed on 2 days to evaluate reproducibility with the optimized manifold. Testing parameters included 1 lot on 04 may and 2 lots on 05 May with 18g of material per test and 29mg gluteraldehyde per gram of hemoglobin (bHB). Testing apparatus in FIG. 28 has a static mixer 3/16″ OD×4 cm length, a T-shaped connector instead of Y-shaped to avoid Glut reflux, valves on retentate tubing for closed system conc./diaf, and continuous N2 sparging.

FIG. 29 is a schematic depicting another embodiment of a polymerization process set up.

FIG. 30 is a series of graphs and images depicting C800 QEX (or equivalent) chromatography gradient optimization 1. Gradient optimization 2 resulted in significant improvement in removal of major 30 KDa impurity along with 75% yield. Loading more than 163 mg bHB/ml resin may be possible.

FIG. 31 is a chart depicting technical specifications for C800 QEX (or equivalent) chromatography gradient optimization 1.

FIG. 32 is a series of graphs and images depicting C800 QEX (or equivalent) chromatography gradient optimization 2. Gradient optimization 2 has a slower gradient and higher protein load compared to optimization 1 (FIG. 30 and FIG. 3). Gradient optimization 2 had a slight amount of bHB in the FT, 80% yield, good efficacy of CIP method (1×), good resolution, and good recovery at 236 mg bHB/ml resin.

FIG. 33 is a chart depicting technical specifications for C800 QEX (or equivalent) chromatography gradient optimization 2.

FIG. 34 is a flow chart depicting C800 QEX (or equivalent) chromatography optimization of CIP of Q sepharose XL.

FIG. 35 is an image of an assembly for C800 QeX chromatorgraphy (or equivalent). This image depicts an assembly and process with 412 ml column (5 cm diameter), 180-220 mg bHB/ml resin, three runs to process C500 1705A, fraction collector to be used for first runs, buffers will be continuously N₂ sparged, and a fraction collector that will be wrapped in an atmosbag inflated with N₂. This gradient method was optimized in April on 2.6cm diameter column.

FIG. 36 is a series of images depicting storage of C500. The product can be stored at 4° C. for up to 4 weeks. Product is bottle sealed in atmosbag filled with N₂ after 3 cycles of vaccum-N₂.

FIG. 37A-FIG. 37E are a series of charts, graphs, and images depicting 10 KDa diafiltration. FIG. 37A is a chart depicting data regarding 10 KDa diafiltraton. FIG. 37B is a plot depicting permeate volume (L) and Flux (LMH) for C5001705A 10 KDa diafilration. FIG. 37C is a plot depicting TMP and Flux (LMH) for C5001705A 10KDa diafilration. FIG. 37D is a schematic of the 10 kDa diafiltration process. FIG. 37E is an image of the 10 KDa diafiltration apparatus. Despite the slight red coloration of the permeate, no bHB was detected by cooximeter. Retentate was filtered by Sartopore 2 sterile MidiCap 0.45 μm+0.2 μm filter.

FIG. 38A-FIG. 38C are a series of charts and graphs depicting 100 KDa diafiltration.

FIG. 38A is a plot of Permeate volume (L) and Permeate bHB concentration (g/dL) for 100 KDa diafiltration. Less than 1% of bHB was measured in the retentate by cooximeter after diafiltration (1.7 g/247g). FIG. 38B is plot of permeate volume (L) and retentate total bHB (%) for 100 KDa diafiltration. FIG. 38C is a chart depicting data from 100 KDa diafiltration process.

FIG. 39 is a series of images of the assembly for the 100 KDa diafiltration process.

FIG. 40 is a schematic of the 100 KDa diafiltration process. The diafiltration process involves (1) Constant N₂ sparging of retentate, permeate, and diafiltration buffer (H₂O) (2) diafiltration H₂O is MilliQ H₂O at <0.005EUml diafiltered with 10 KDa membrane (3) Addition of diafiltration buffer is performed through a T fitting with a static mixer directly in the retentate tube to improve the homogeneity of the retentate without using magnetic stirrer. (4) Permeate flow control with peristaltic pump to prevent formation of gel layer and flux reduction and to bridge with large pilot scale. (5) Brief passage of the feed through 40° C. heat exchanger before entering the membrane which promotes increase in the proportion of the transient dimeric bHB form to improve diafiltration efficacy and yield.

FIG. 41 is a schematic depicting hollow fiber next batch blood wash. A 0.65 μm hollow fiber will be available for next batch. The set up will include permeate flow control.

FIG. 42A-FIG. 42C are a series of images and charts depicting blood wash and lysis. FIG. 42A is a chart depicting data for blood wash and lysis processes. FIG. 42B is an image of the blood wash and lysis apparatus. FIG. 42C is a more complete image of the blood wash and lysis process apparatus. For the wash a hollow fiber cartridge was not available. Red cells are washed by centrifugation. Blood is diluted 1:1 in Citrate saline (CSB) and centrifuged. Cell pellet is resuspended in CSB and centrifuged three times (total of four centrifugations). For the lysis a 1:1 dilution in H₂O with static mixing. Centrifugation 14000×g to remove cell debris.

### DETAILED DESCRIPTION OF THE INVENTION

More than 99% of the cells in blood are red blood cells. The major function of red blood cells is to transport hemoglobin, which in turn carries oxygen from lungs to the tissues and CO2 from the tissues to the lungs. Normal red blood cells contain approximately 34 grams of hemoglobin per 100 ml of cells. Each gram of hemoglobin is capable of combining with approximately 1.33 ml of oxygen. In bovine blood the concentration of hemoglobin (bHB) in g/dL is 10.1 and with a volume of 2.96 L of blood this amounts to 299 g of bHB. Thus, bovine blood is a viable option for large-scale hemoglobin recovery.

### Separation System for Protein Purification

For example in some embodiments the separation system used for protein purification is a CARR Centritech UniFuge system from PneumaticScaleAngelus (or equivalent system). The UniFuge system utilizes a gamma irradiated, single-use module that requires NO CIP and NO SIP. All process contact surfaces are easy to install and are 100% replaceable after each run. Low shear harvesting of mammalian and insect cells is possible, and minimal reduction in viability of recovered cells is achievable. Since the cells are not lysed, production of cell debris in the centrifuge is minimized, making the UniFuge an excellent choice for both cell recovery or centrate clarification. UniFuge modules are readily tube welded to your single-use bioreactor connections. The UniFuge is completely automated with flexible cycle parameter entry. The feed suspension is gently pumped to the module and the cells settle to the outer radius while the clear supernatant is continuously discharged. Once the module has filled with cells, the controller stops the rotor and discharges the cells. This cycle is repeated until the bioreactor volume has been processed.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: 2.3.S.2.2. Description of Manufacturing Process and Process Controls for Small Batch Oxyply Drug Substance Manufacture

### Blood Collection

Bovine blood is obtained from farms affiliated with the Universite de Montreal School of Veterinary Medicine. The animals are continuously observed through the school's documented health program.

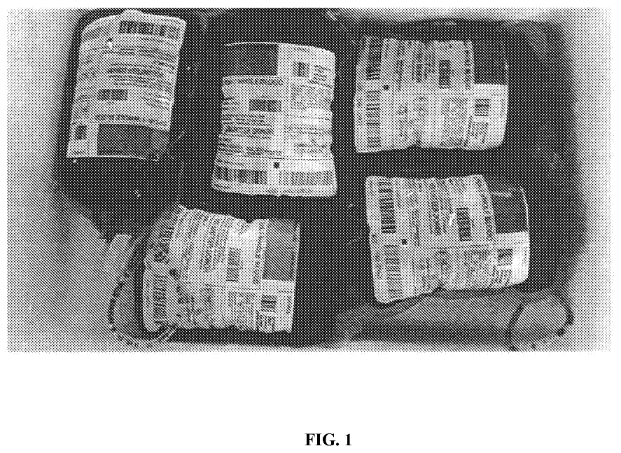

Blood in volumes of up to one (1) liter are obtained per animal via venipuncture from the coccygeal vein. Collection is made using a 500 milliliters (mL) Double Blood Pack collection system (FIG. 1, Fenwal, part number 4R3429, Lake Zurich, Ill.). Bags contain CPD anticoagulant and are equipped with a satellite container and sterile needle/tubing sampling system. The cow's tail is raised and a 16 gauge needle is inserted about one-half inch deep and perpendicular to the tail and the underside, midline and three to six inches from the base of the tail. Blood is collected by into the bag by gravity, until 450-500 mL are obtained. Immediately after collection, the bags are placed on ice and transported to the processing facility (e.g. Biodextris).

### Cell Washing

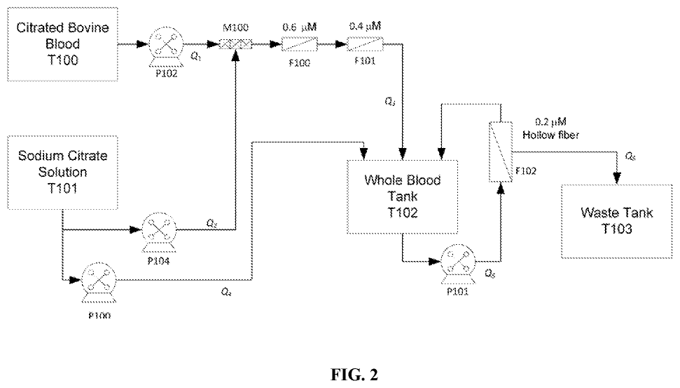

Collected blood is washed according the process shown in FIG. 2. Blood, 3-5 liters (L), from multiple collections performed within the previous 24 hours, is transferred to a single Mobius 5 L flexible bag (T100) using a peristaltic pump. 50 L Sodium Citrate Solution (7.9 g/L sodium chloride and 6.0 g/L sodium citrate dihydrate with purified water) is prepared in a sterile mixing tank and depyrogenated by passage through a 10 kDa membrane filter into a 50 L flexible bag (T101). Citrated blood is pumped into a static in-line mixer at a flow rate of 200 mL-min⁻¹, simultaneously with Sodium Citrate Solution at a flow rate of 280 mL-min⁻¹. The mixture is directed through sequential 0.6 μM and 0.4 μM depth filtration membranes and into a 20 L flexible bag (T102). When bag T102 contains 5 L of filtered blood, the washing process is initiated by recirculation through a 0.2 μM hollow fiber membrane at a rate of 1 L-min⁻¹. Transmembrane pressure is adjusted to 15 psi, allowing for an average permeate flow rate of 300 mL-min⁻¹. Cell washing, by diafilitration, is initiated by pumping Sodium Citrate Solution into bag T102 at a flow rate of 300 mL-min⁻¹, and continues until the cells are washed with 7 volumes. The diafiltration permeate is directed into a 50 L flexible waste bag (T103). Diafiltration continues until permeate equivalent to 7 blood volumes is collected. Examples of parts used for cell washing process is given in TABLE 1 below.

TABLE 1

| ID | Part | Manufacturer |
|---|---|---|
| T100 | Mobius 5 L | Merck Millipore |
| T101 | Mobius 50 L | Merck Millipore |
| T102 | Mobius 20 L | Merck Millipore |
| T103 | Mobius 50 L | Merck Millipore |
| P100 | Stainless Digital Process Pump | Masterflex |
| P101 | Stainless Digital Process Pump | Masterflex |
| F100 | | Sartorius |
| F101 | | |
| F102 | | |
| V100 | | |
| M100 | Static Mixer | Koflo |

An alternate to this process is to carry out this step using larger scale equipment or to install a centrifuge and carry out the c500 steps at 25 L. The current set-up is designed to limit tank (bag size) to 50 L so that the bag can fit on a moveable rack.

### Cell Lysis

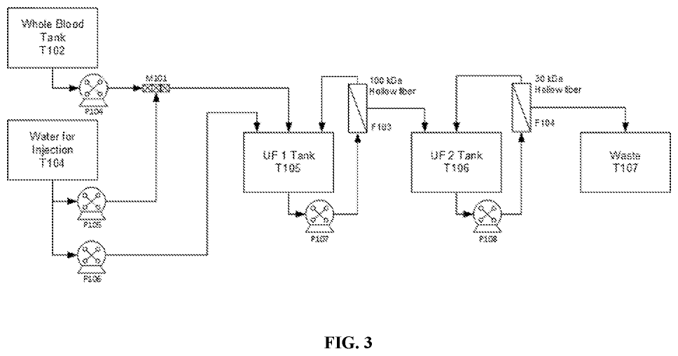

Hemoglobin is liberated from bovine red blood cells when cells are lysed by a rapid decrease in osmotic pressure. Cell lysis and sequential diafiltration across 100 kDa and 30 kDa membranes is carried out as shown in FIG. 3. Citrated Whole Blood is pumped into a static in-line mixer at a flow rate of 250 mL-min⁻¹, simultaneously with Water for Injection at a flow rate of 250 mL-min⁻¹ into a 10 L flexible bag (T105 ). When T105 is filled with 2.0-2.5 L of diluted Whole Blood, recirculation is initiated through the 100,000 kDa hollow fiber membrane cartridge (F103) at a flow rate of 1000 mL-min⁻¹. The permeate is directed to a 5 L flexible bag (T106). When 1.0-1.5 L of permeate has accumulated in T106, recirculation through the 30,000 kDa membrane (F104) is initiated at a flow rate of 1000 mL-min⁻¹. The F104 permeate is directed to waste. Pumps 104 and 105 are stopped when the volume of Whole Blood (T102) is less than 250 mL. Diafiltration is then started by pumping WFI directly into T105 at a flow rate of for instance 250 mL-min⁻¹ and continues until the hemoglobin concentration in the 100,000 kDa permeate is less than 0.2 mg-mL⁻¹, corresponding to approximately 25-30 L diafiltration volume. Examples of parts used for cell lysis process is given in TABLE 2 below.

TABLE 2

| ID | Part | Manufacturer |
|---|---|---|
| T102 | Mobius 20 L | Merck Millipore |
| T104 | Mobius 50 L | Merck Millipore |
| T105 | Mobius 10 L | Merck Millipore |
| T106 | Mobius 5 L | Merck Millipore |
| T107 | Mobius 50 L | Merck Millipore |
| P104 | Stainless Digital Process Pump | Masterflex |
| P105 | Stainless Digital Process Pump | Masterflex |
| P106 | Stainless Digital Process Pump | Masterflex |
| P107 | Stainless Digital Process Pump | Masterflex |
| P108 | Stainless Digital Process Pump | Masterflex |
| F100 | | Sartorius |
| F101 | | |
| F102 | | |
| M101 | Static Mixer | Koflo |

### Deoxygenation of Hemoglobin Solution

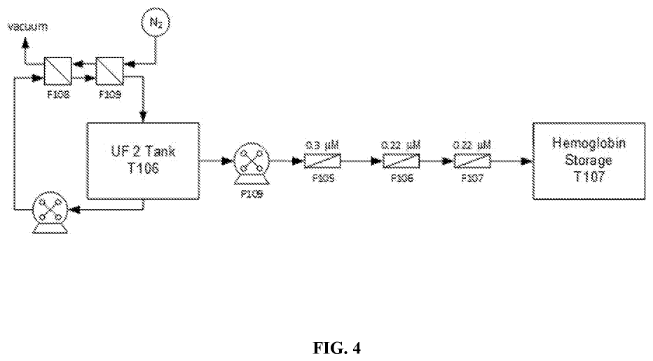

The hemoglobin solution is stabilized by removing oxygen and filtered for storage as an intermediate using a process depicted in FIG. 4. Initially, the hemoglobin solution is pumped through two Liquicell Membranes aligned in series at a flow rate of 500 ml-min⁻¹, with a counter-current flow of nitrogen at 75 psi. Deoxygenation continues until the dissolved oxygen reading is below 0.02 mg-mL⁻¹. When sufficient deoxygenation is achieved, the hemoglobin solution is filtered by pumping through a 0.3 μM and two 0.22 μM depth filters into a 5 L flexible bag. Filtered hemoglobin can be stored for up to 2 weeks before further processing. Examples of parts used for hemoglobin filtration-deoxygenation process is given in TABLE 3 below.

TABLE 3

| ID | Part | Manufacturer |
|---|---|---|
| T106 | Mobius 5 L | Merck Millipore |
| T107 | Mobius 5 L | Merck Millipore |
| P109 | Stainless Digital Process Pump | Masterflex |
| P110 | Stainless Digital Process Pump | Masterflex |
| F105 | 0.3 μM depth filter | Sartorius |
| F106 | 0.22 μM depth filter | |
| F107 | 0.22 μM depth filter | |
| F108 | Liquicel gas exchange membrane | 3M |
| F109 | Liquicel gas exchange membrane | 3M |

### Chromatography

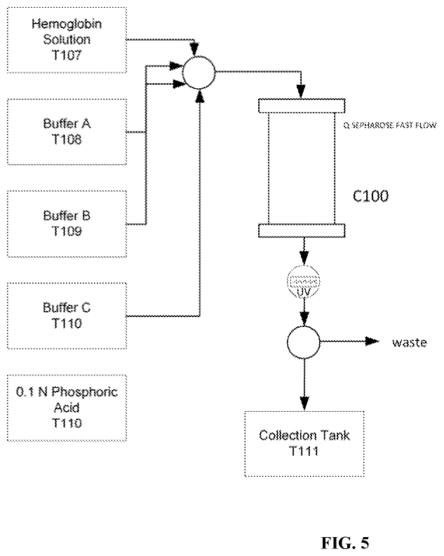

Chromatography is used to further purify the hemoglobin solution and reduce non-specific blood cell components (process depicted FIG. 5). This is performed using a GE Akta Biopilot chromatography system equipped with a GE Healthcare XK borosilicate column (5 cm i.d.×100 cm length) packed with Q Sepharose Fast Flow (GE Healthcare) to a bed height of 70±5 cm. Buffers are prepared using Water for Injection and filtered through a 10 kDa membrane to further reduce pyrogen content. Buffers are: (1) Buffer A; 2.42 g-L⁻¹ tris base adjusted to pH 9.0±0.1 with acetic acid, (2) Buffer B; 6.05 g-L⁻¹ Tris base adjusted to pH 7.0±0.1 with acetic acid and (3) Buffer C; 2.42 g-L⁻¹ Tris base and 58.38 g-L-1 NaCl adjusted to pH 8.9±0.1 with acetic acid.

Prior to the chromatographic operation, five complete buffer cycles are run through freshly packed Q Sepharose columns. Chromatography is carried out at a flow rate of 125 mL-min⁻¹. Hemoglobin Solution, 1 L containing 130±10 mg-mL⁻¹ hemoglobin, is initially loaded onto the column followed by the creation of a pH gradient formed by adding equal volumes of Buffer A and Buffer B. Protein eluting from the column is measured by UV absorbance at 280 nm. When absorbance of the eluate is falls below 0.05 AU, the column pH is increased by elution with 100% Buffer B. Hemoglobin elutes during this portion of the chromatographic run. The hemoglobin fraction is collected into a 20 L flexible bag (T111) when the absorbance reaches 0.43 AU and terminates when the absorbance falls below 0.05 AU. Following elution of hemoglobin, 3 L of Buffer C is pumped through the column to elute tightly bound constituents.

The column is cleaned between each chromatographic run using 0.2 N phosphoric acid followed by two complete buffer cycles. Columns are stored in 0.2 N phosphoric acid if another run is not to be initiated within 24 hours. Examples of parts used for chromatography process is given in TABLE 4 below.

TABLE 4

| ID | Part | Manufacturer |
|---|---|---|
| T107 | Mobius 5 L | Merck Millipore |
| T108 | Mobius 50 L | Merck Millipore |
| T109 | Mobius 50 L | Merck Millipore |
| T110 | Mobius 50 L | Merck Millipore |
| T111 | Mobius 20 L | Merck Millipore |
| | Q Sepharose Fast Flow resin | GE |
| C100 | BioPilot chromatograpy System | GE |

### Deoxygenation

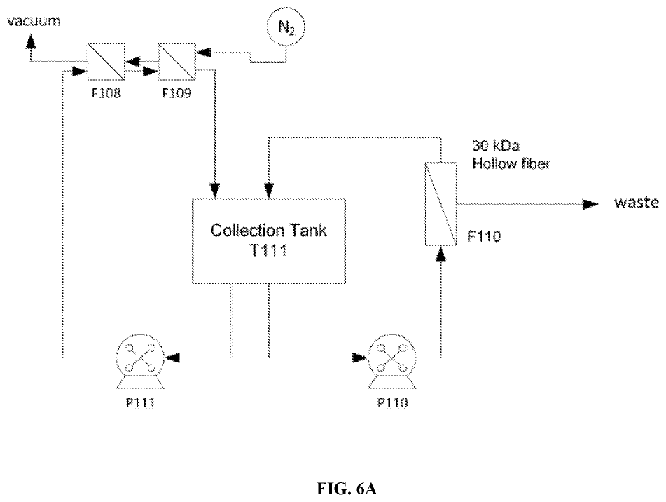

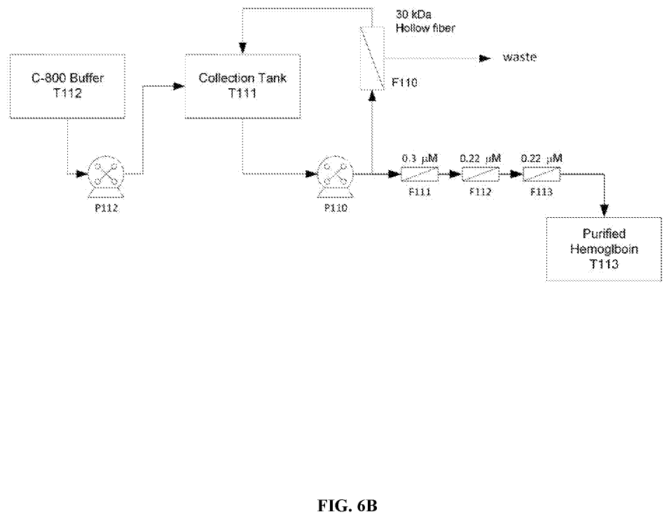

Purified Hemoglobin is deoxygenated to increase stability as shown in FIG. 6A-FIG. 6B. Purified fractions from the anion exchange chromatography step are concentrated to 11.0±1 mg-mL¹ by filtration through a 30,000 Da hollow-fiber membrane (F110). When the desired hemoglobin concentration is reached, the Purified Hemoglobin is deoxygenated by passage through two Liquicell Membranes (F108, F109) aligned in series at a flow rate of 500 ml-min⁻¹, with a counter-current flow of nitrogen at 75 psi. Deoxygenation continues until the dissolved oxygen reading is below 0.02 mg-mL⁻¹.

The deoxygenated Purified Hemoglobin is subsequently diafiltered against six volumes of storage buffer by pumping through a 30,000 Da hollow-fiber membrane (F110). The composition of the storage buffer is 2.63 g-L⁻¹ tribasic sodium phosphate dodecahydrate, 7.0 g-L-1 dibasic sodium phosphate heptahydrate and 2.0 g-L⁻¹ acetylcysteine. When the buffer exchange is complete the solution is filtered by pumping through a 0.5 μM and two 0.22 μM depth filters into a 5 L flexible bag (T113). The Purified Hemoglobin can be stored in a Nitrogen Glove Box for up to 60 days at room temperature (17-23° C.) before further processing. Examples of parts used for deoxygenation process is given in TABLE 5 below.

TABLE 5

| ID | Part | Manufacturer |
|---|---|---|
| T107 | Mobius 5 L | Merck Millipore |
| T108 | Mobius 50 L | Merck Millipore |
| T109 | Mobius 50 T | Merck Millipore |
| T110 | Mobius 50 L | Merck Millipore |
| T111 | Mobius 20 L | Merck Millipore |
| | Q Sepharose Fast Flow resin | GE |
| C100 | BioPilot chromatograpy System | GE |

### Polymerization

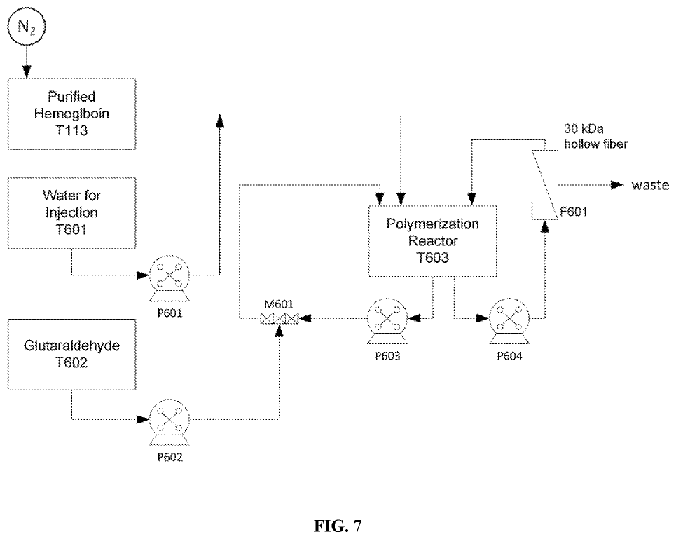

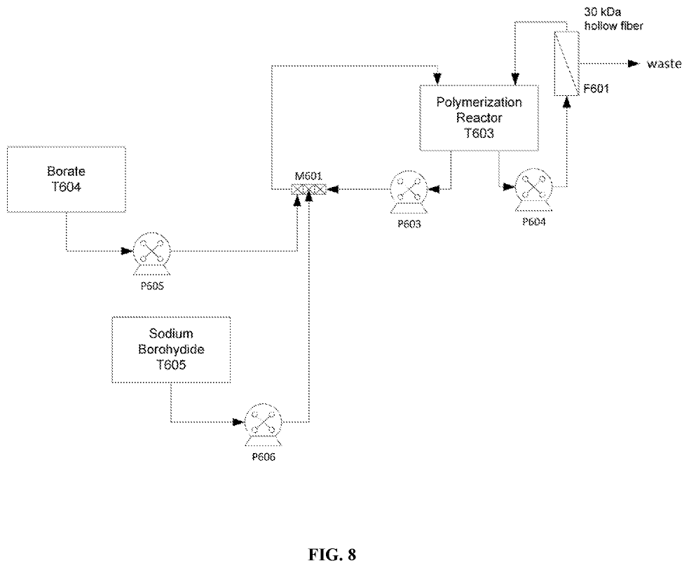

Purified Hemoglobin is polymerized by cross-linking with glutaraldehyde using the process depicted in FIG. 7. Purified Hemoglobin (4-5 L, 110 g/L) is transferred from Storage Tank (T113) by under nitrogen pressure to a 20 L temperature controlled wave bag (T603). Water for Injection is pumped through the Purified Hemoglobin transfer line into T603 to reduce the hemoglobin concentration to 40 g/L. The temperature of the diluted Hemoglobin solution is then raised to 42±2° C. Glutaraldehyde solution is prepared at a concentration of 6.2 g/L in a temperature controlled Wave bag (T602) and heated to 42±2 □C. The Glutaraldehyde solution is pumped into T603 at a rate of 10 mL/min until the ratio of glutaraldehyde to hemoglobin is approximately 0.029:1. The glutaraldehyde is added through a static mixer (M601) in a recirculation loop to ensure rapid and homogeneous mixing with the hemoglobin solution. When the addition of glutaraldehyde is completed, the temperature of the reaction mixture is cooled to 22±2° C. and the solution is concentrated by diafiltration through a 30,000 Da hollow-fiber membrane (F601) to a hemoglobin concentration of 80±5 g/L.

Glutaraldehyde-hemoglobin bonds are stabilized by reduction with sodium borohydride as summarized in FIG. 8. Sodium borohydride decomposes in aqueous solution at neutral pH to form molecular hydrogen and sodium borate. Diafiltration of polymerised hemoglobin with sodium borate buffer is carried out to stabilize sodium borohydride and limit hydrogen gas formation. Borate buffer is composed of 4.58 g/L sodium borate decahydrate and 0.91 g/L sodium hydroxide in Water for Injection.

The buffer is filtered through a 10,000 Da membrane to reduce pyrogen content and is stored in a 20 L flexible bag (T605). The borate buffer is pumped into T603, through the recirculation loop, initially at a flow rate of 250 mL/min. Simultaneously, the polymerized hemoglobin solution is diafiltered by pumping through a 30,000 Da hollow fiber membrane at a flow rate of 1,000 mL/min. The borate addition flow rate is adjusted to equal that of the diafiltration permeate rate, approximately 250 mL/min. Diafiltration with borate buffer continues until the volume corresponding to 3 times that of the polymerized hemoglobin solution have been added.

Sodium borohydride solution is comprised of 9.45 g/L sodium borohydride, 4.58 g/L sodium borate decahydrate and 0.91 g/L sodium hydroxide in Water for Injection. The solution is filtered through a 10,000 Da membrane to reduce pyrogen content and stored in a 2 L flexible bag (T606). Sodium Borohydride solution (0.6 L) is pumped into T603, through the recirculation loop, initially at a flow rate of 7 mL/min and the temperature of T603 controlled at 20±2° C. The borohydride reaction continues for 60 minutes after all the solution has been added, with continuous recirculation of the polymerized hemoglobin solution.

The stabilized polymerised hemoglobin solution is concentrated across the 30 kD ultrafiltration membrane (F601) to a hemoglobin concentration of 100±5 g/L. Boron containing components (sodium borate/sodium borohydride) are removed and the pH reduced to 8.0-8.4 by diafiltration of the polymerised hemoglobin across 30 kD ultrafiltration membrane (F601) with Diafiltration Solution A (6.67 g/L sodium chloride, 0.30 g/L potassium chloride, 0.20 g/L calcium chloride dihydrate, 0.445 g/L sodium hydroxide, 2.02 g/L N-acetyl-L-cysteine, 3.07 g/L sodium lactate, pH=4.9-5.1). Examples of parts used for the polymerization process is given in TABLE 6 below.

TABLE 6

| ID | Part | Manufacturer |
|---|---|---|
| T113 | Mobius 5 L | Merck Millipore |
| T601 | Mobius 50 L | Merck Millipore |
| T602 | Mobius 50 L | Merck Millipore |
| T603 | Mobius 50 L | Merck Millipore |
| T604 | Mobius 20 L | Merck Millipore |
| T605 | Q Sepharose Fast Flow resin | GE |
| P601 | Stainless Digital Process Pump | Masterflex |
| P602 | Stainless Digital Process Pump | Masterflex |
| P603 | Stainless Digital Process Pump | Masterflex |
| P604 | Stainless Digital Process Pump | Masterflex |
| P605 | Stainless Digital Process Pump | Masterflex |
| P606 | Stainless Digital Process Pump | Masterflex |
| M601 | Static Mixer | Kobi |

### Sterile Filtration

Final polymerised haemoglobin solution is filtered through a 0.5 μm depth filter, a sterilizing grade 0.2 μm membrane filter, and a 2nd sterilizing grade 0.2 μm membrane filter into a 275-liter steam sanitized portable bulk holding tank. The bulk holding tank is stored under nitrogen until use.

### Example 2: Description of Manufacture Process and Process Controls for Bulk Manufacturing of Oxyply Drug Substance

### Introduction

The manufacture of OxyPly bulk drug substance involves the following major steps;
1. Blood Collection-bovine blood collected in sodium citrate anticoagulant

### Blood Collection

Bovine blood is obtained from farms affiliated with the Universite de Montréal School of Veterinary Medicine. The animals are continuously observed through the school's documented health program.

Blood in volumes of up to one (1) liter are obtained per animal via venipuncture from the coccygeal vein. Collection is made using a 500 mL Double Blood Pack collection system (Fenwal, part number 4R3429, Lake Zurich, Ill.). Bags contain CPD anticoagulant and are equipped with a satellite container and sterile needle/tubing sampling system. The cow's tail is raised and a 16 gauge needle is inserted about one-half inch deep and perpendicular to the tail and the underside, midline and three to six inches from the base of the tail. Blood is collected by into the bag by gravity, until 450-500 mL are obtained. Immediately after collection, the bags are placed on ice and transported to the processing facility.

### Cell Washing

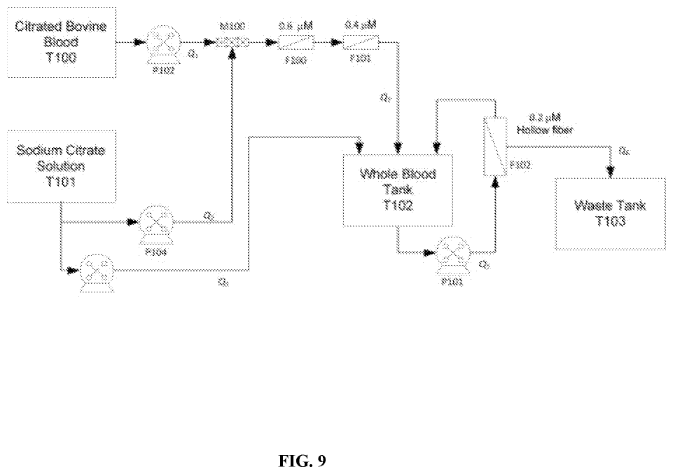

Collected blood is washed according the process shown FIG. 9. Blood, 15-20 L, from multiple collections performed within the previous 24 hours, is transferred to a single 20 L GE Ready Circuit flexible bag (T100) using a peristaltic pump. 200 L Sodium Citrate Solution (7.9 g/L sodium chloride and 6.0 g/L sodium citrate dihydrate with purified water) is prepared in a sterile mixing tank and depyrogenated by passage through a 10,000 Da membrane filter into a 200 L Ultra Low-Density Polyethylene (ULDP) single use bag (T101). Citrated blood is pumped into a static in-line mixer at a flow rate of 500 mL-min⁻¹, simultaneously with Sodium Citrate Solution at a flow rate of 700 mL-min⁻¹. The mixture is directed through sequential 0.6 μM and 0.4 μM depth filtration membranes and into a 50 L ULDP single use bag (T102). When bag T102 contains 10 L of filtered blood, the washing process is initiated by recirculation through a 0.2 μM hollow fiber membrane at a rate of 2 L-min⁻¹. Transmembrane pressure is adjusted to 15 psi, allowing for an average permeate flow rate of 500 mL-min⁻¹. Cell washing, by diafilitration, is initiated by pumping Sodium Citrate Solution into bag T102 at a flow rate of 500 mL-min⁻¹, and continues until the cells are washed with 7 diafiltration volumes. The diafiltration permeate is directed into a 200 L ULDP single use bag (T103). Diafiltration continues until permeate equivalent to 7 blood volumes is collected.

Examples of parts used for cell wash process is given in TABLE 7 below, and examples of parts used for cell wash in-process testing is given in TABLE 8 below.

TABLE 7

| ID | Part | Manufacturer |
|---|---|---|
| T100 | Ready Circuit 20 L | GE Healthcare |
| T101 | Xcellerex XDM 200 L | GE Healthcare |
| T102 | Xcellerex XDM 50 L | GE Healthcare |
| T103 | Xcellerex XDM 200 L | GE Healthcare |
| P100 | Stainless Digital Process Pump | Masterflex |
| P101 | Stainless Digital Process Pump | Masterflex |
| F100 | 0.6 μM depth filter | Sartorius |
| F101 | 0.4 μM depth filter | Sarlorias |
| F102 | 0.2 μM hollow-fiber | Sartorius |
| M100 | Static Mixer | Koflo |

TABLE 8

| Test Material/Parameter | Measurement |
|---|---|
| Citrated Bovine Blood | Total Hemoglobin (Hgb) |
| Sodium Citrate Solution | LAL |
| F102 Permeate | Protein (UV280) |

### Cell Lysis

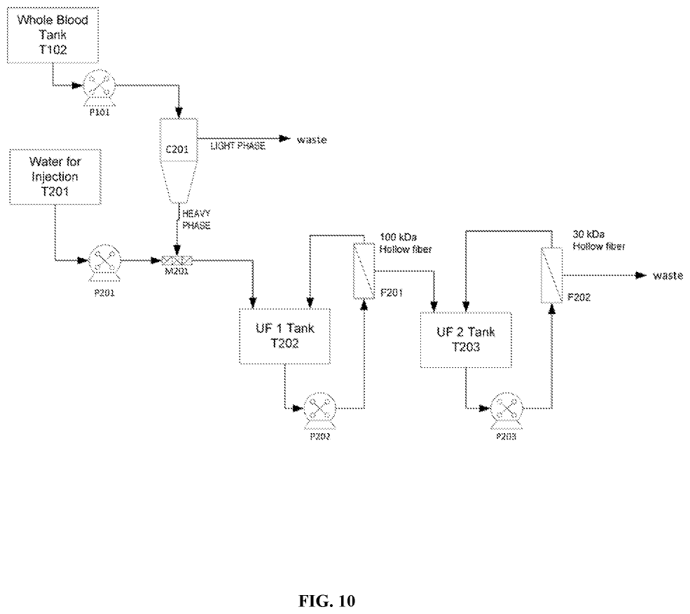

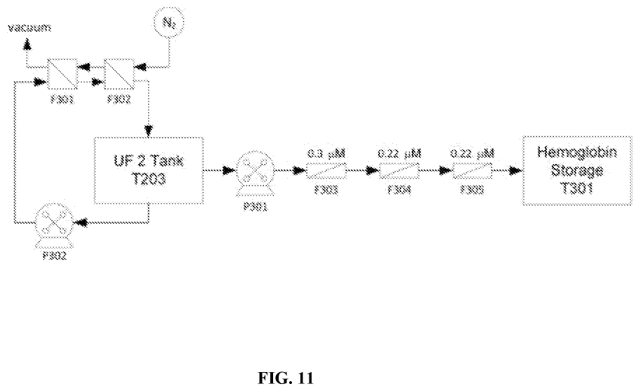

Red blood cells are separated from white blood cells and platelets by centrifugation and the hemoglobin liberated from red blood cells when cells are lysed by a rapid decrease in osmotic pressure as shown in FIG. 10. Washed blood cells are pumped into a tubular bowl centrifuge (C201) operating at 13,500×g. Red blood cells contained in the heavy phase are directed through a static mixer (M201), where they are diluted 2-fold with Water for Injection, and into a 20 L GE Ready Circuit flexible bag (T202). When T202 is filled with at least 10 L of diluted Whole Blood, recirculation is initiated through the 100,000 kDa hollow fiber membrane cartridge (F201) at a flow rate of 1000 mL-min-1. The permeate is directed to a 20 L GE Ready Circuit flexible bag (T203). When 15 L of permeate has accumulated in T203, recirculation through the 30,000 kDa membrane (F202) is initiated at a flow rate of 1000 mL-min-1. The F202 permeate is directed to waste. Diafiltration through the 100,000 Da membrane (F201) continues until the hemoglobin concentration in the permeate is less than 0.2 mg/mL, indicating that most of the liberated hemoglobin has been extracted. This corresponds to approximately 15-20 diafiltration volumes. corresponding to approximately 25-30 L diafiltration volume. Hemoglobin, separated from the cell debris by 100,000 Da filtration, is concentrated by filtration against a 30,000 kDa membrane. The 100,000 Da and 30,000 Da steps are carried out in a continuous process. The 30,000 Da filtration is stopped when the hemoglobin concentration is in the range of 90-110 g/L.

Examples of parts used for cell lysis process is given in TABLE 9 below, and examples of parts used for cell lysis in-process testing is given in TABLE 10 below.

TABLE 9

| ID | Part | Manufacturer |
|---|---|---|
| T102 | Xcellerex XDM 50 L | GE Healthcare |
| T201 | Xcellerex XDM 200 L | GE Healthcare |
| T202 | 20 L Ready Circuit | GE Healthcare |
| T203 | 20 L Ready Circuit | GE Healthcare |
| T204 | Xcellerex XDM 200 L | GE Healthcare |
| P101 | Stainless Digital Process Pump | Masterflex |
| P201 | Stainless Digital Process Pump | Masterflex |
| P202 | Stainless Digital Process Pump | Masterflex |
| P203 | Stainless Digital Process Pump | Masterflex |
| F201 | 100 kDa hollow-fiber | Sartorius |
| F202 | 30 kDa hollow-fiber | Sartorius |
| M201 | Static Mixer | Koflo |

TABLE 10

| Test Material/Parameter | Measurement |
|---|---|
| Citrated Bovine Blood | Total Hemoglobin (Hgb) |
| Water for Injection | LAL |
| 100,000 Da(F201) Permeate | Total Hemoglobin (Hgb) |
| 30,000 Da(F201) Retentate | Total Hemoglobin (Hgb) |

### Deoxygenation of Hemoglobin Solution

The hemoglobin solution is stabilized by removing oxygen and filtered for storage as an intermediate using a process depicted in FIG. 11. Initially, the hemoglobin solution is pumped through two Liquicell Membranes aligned in series at a flow rate of 500 ml-min⁻¹, with a counter-current flow of nitrogen at 75 psi. Deoxygenation continues until the dissolved oxygen reading is below 0.02 mg/mL. When sufficient deoxygenation is achieved, the hemoglobin solution is filtered by pumping through a 0.3 μM and two 0.22 μM depth filters into a 20 L GE Ready Circuit flexible bag (T301). Filtered hemoglobin can be stored for up to 2 weeks before further processing.

Examples of parts used for hemoglobin filtration-deoxygenation process is given in TABLE 11 below, and examples of parts used for hemoglobin filtration-deoxygenation in-process testing is given in TABLE 12 below.

TABLE 11

| ID | Part | Manufacturer |
|---|---|---|
| T202 | 20 L Ready Circuit | GE Healthcare |
| T301 | 20 L Ready Circuit | GE Healthcare |
| P109 | Stainless Digital Process Pump | Masterflex |
| P110 | Stainless Digital Process Pump | Masterflex |
| F105 | 0.3 μM depth filter | Sartorius |
| F106 | 0.22 μM depth filter | Sartorius |
| F107 | 0.22 μM depth filter | Sartorius |
| F108 | Liquicel gas exchange membrane | 3M |
| F109 | Liquicel gas exchange membrane | 3M |

TABLE 12

| Test Material/Parameter | Measurement |
|---|---|
| Washed hemoglobin (T203) | Dissolved oxygen |
| | Total Hgb |
| | Met-Hgb |
| | Oxy-Hgb |
| Hemoglobin Storage | Dissolved oxygen |
| | Total Hgb |
| | Met-Hgb |
| | Oxy-Hgb |

### Chromatography

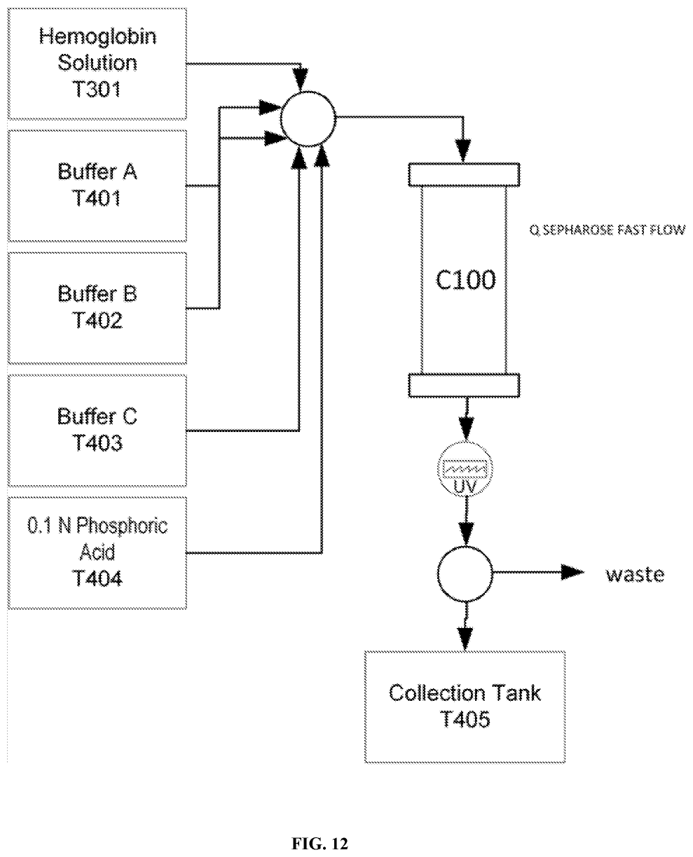

Chromatography is used to further purify the hemoglobin solution and reduce non-specific blood cell components (process depicted in FIG. 12). This is performed using a GE Akta Biopilot chromatography system equipped with a GE Healthcare XK borosilicate column (5 cm i.d.×100 cm length) packed with Q Sepharose Fast Flow (GE Healthcare) to a bed height of 70 ±5 cm. Buffers are prepared using Water for Injection and filtered through a 10 kDa membrane to further reduce pyrogen content. Buffers are: (1) Buffer A; 2.42 g-L⁻¹ tris base adjusted to pH 9.0±0.1 with acetic acid, (2) Buffer B; 6.05 g-L⁻¹ Tris base adjusted to pH7.0±0.1 with acetic acid and (3) Buffer C; 2.42 g-L⁻¹ Tris base and 58.38 g-L⁻¹ NaCl adjusted to pH 8.9±0.1 with acetic acid.

Prior to the chromatographic operation, five complete buffer cycles are run through freshly packed Q Sepharose columns. Chromatography is carried out at a flow rate of 125 mL-min⁻¹. Hemoglobin Solution, 1 L containing 130±10 mg-mL⁻¹ hemoglobin, is initially loaded onto the column followed by the creation of a pH gradient formed by adding equal volumes of Buffer A and Buffer B. Protein eluting from the column is measured by UV absorbance at 280 nm. When absorbance of the eluate is falls below 0.05 AU, the column pH is increased by elution with 100% Buffer B. Hemoglobin elutes during this portion of the chromatographic run. The hemoglobin fraction is collected into a 20 L GE Ready Circuit single use bag (T405) when the absorbance reaches 0.43 AU and terminates when the absorbance falls below 0.05 AU. Following elution of hemoglobin, 3 L of Buffer C is pumped through the column to elute tightly bound constituents.

The column is cleaned between each chromatographic run using 0.2 N phosphoric acid followed by two complete buffer cycles. Columns are stored in 0.2 N phosphoric acid if another run is not to be initiated within 24 hours.

Examples of parts used for the chromatography process is given in TABLE 13 below, and examples of parts used for chromatography in-process testing is given in TABLE 14 below.

TABLE 13

| ID | Part | Manufacturer |
|---|---|---|
| T301 | 20 L Ready Circuit | GE Healthcare |
| T401 | 50 L Ready Circuit | GE Healthcare |
| T402 | 50 L Ready Circuit | GE Healthcare |
| T403 | 50 L Ready Circuit | GE Healthcare |
| T404 | 50 L Ready Circuit | GE Healthcare |
| | Q Sepharose Fast Flow resin | GE Healthcare |
| C100 | BioPilot chromatograpy System | GE Healthcare |

TABLE 14

| Test Material/Parameter | Measurement |
|---|---|
| Column eluate | UV280 |
| Chromatography Buffers | LAL |

### Deoxygenation

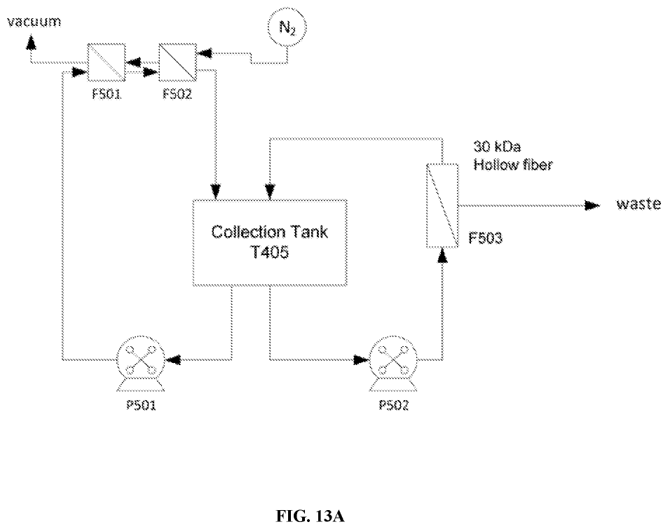

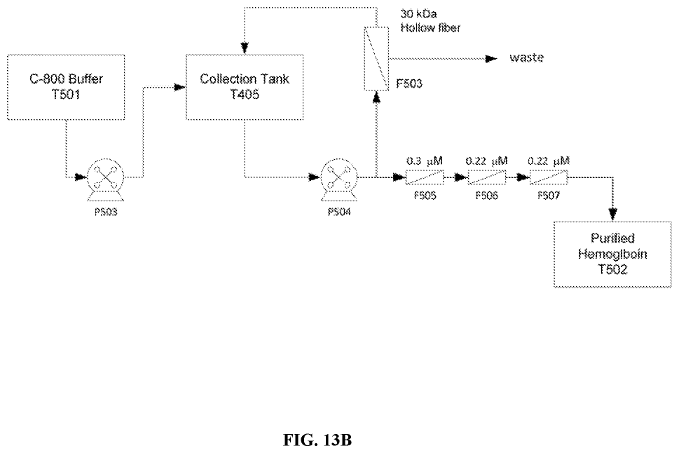

Purified Hemoglobin is deoxygenated to increase stability as shown in FIG. 13A and FIG. 13B. Purified fractions from the anion exchange chromatography step are concentrated to 11.0±1 mg-mL⁻¹ by filtration through a 30,000 Da hollow-fiber membrane (F503). When the desired hemoglobin concentration is reached, the Purified Hemoglobin is deoxygenated by passage through two Liquicell Membranes (F501, F502) aligned in series at a flow rate of 500 ml-min-1, with a counter-current flow of nitrogen at 75 psi. Deoxygenation continues until the dissolved oxygen reading is below 0.02 mg/mL.

The deoxygenated Purified Hemoglobin is subsequently diafiltered against six volumes of storage buffer by pumping through a 30,000 Da hollow-fiber membrane (F110). The composition of the storage buffer is 2.63 g-L⁻¹ tribasic sodium phosphate dodecahydrate, 7.0 g-L-1dibasic sodium phosphate heptahydrate and 2.0 g-L⁻¹acetylcysteine. When the buffer exchange is completed the solution is filtered by pumping through a 0.5 μM and two 0.22 μM depth filters into a 20 L GE Ready Circuit single use bag (T501). The Purified Hemoglobin can be stored in a Nitrogen Glove Box for up to 60 days at room temperature (17-23° C.) before further processing.

Examples of parts used for the deoxygenation process is given in TABLE 15 below, and examples of parts used for deoxygenation in-process testing is given in TABLE 16 below.

TABLE 15

| ID | Part | Manufacturer |
|---|---|---|
| T405 | 20 L Ready Circuit | GE Healthcare |
| T501 | 50 L Ready Circuit | GE Healthcare |
| T502 | 20 L Ready Circuit | GE Healthcare |
| F501 | Liquicel gas exchange membrane | 3M |
| F502 | Liquicel gas exchange membrane | 3M |
| F503 | 30,000 Da hollow fiber | Sartorius |
| F504 | 0.3 uM depth filtration cartridge | Sartorius |
| F505 | 0.22 uM depth filtration cartridge | Sartorius |
| F506 | 0,22 uM depth filtration cartridge | Sartorius |

TABLE 16

| Test Material/Parameter | Measurement |
|---|---|
| Column eluate | UV280 |
| Chromatography Buffers | LAL |

### Polymerization

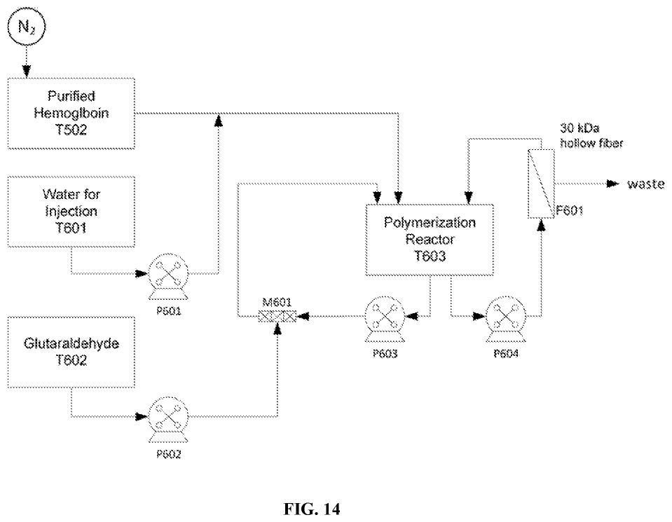

Purified Hemoglobin is polymerized by cross-linking with glutaraldehyde using the process depicted in FIG. 14. Purified Hemoglobin (4-5 L, 110 g/L) is transferred from Storage

Tank (T501) by under nitrogen pressure to a 20 L temperature controlled Wave bag (T603). Water for Injection is pumped through the Purified Hemoglobin transfer line into T603 to reduce the hemoglobin concentration to 40 g/L. The temperature of the diluted Hemoglobin solution is then raised to 42±2° C. Glutaraldehyde solution is prepared at a concentration of 6.2 g/L in a temperature controlled Wave bag (T602) and heated to 42±2° C. The glutaraldehyde solution is pumped into T603 at a rate of 10 mL/min until the ratio of glutaraldehyde to hemoglobin is approximately 0.029:1. The glutaraldehyde is added through a static mixer (M601) in a recirculation loop to ensure rapid and homogeneous mixing with the hemoglobin solution. When the addition of glutaraldehyde is completed, the temperature of the reaction mixture is cooled to 22±2° C. and the solution is concentrated by diafiltration through a 30,000 Da hollow-fiber membrane (F601) to a hemoglobin concentration of 80±5 g/L.

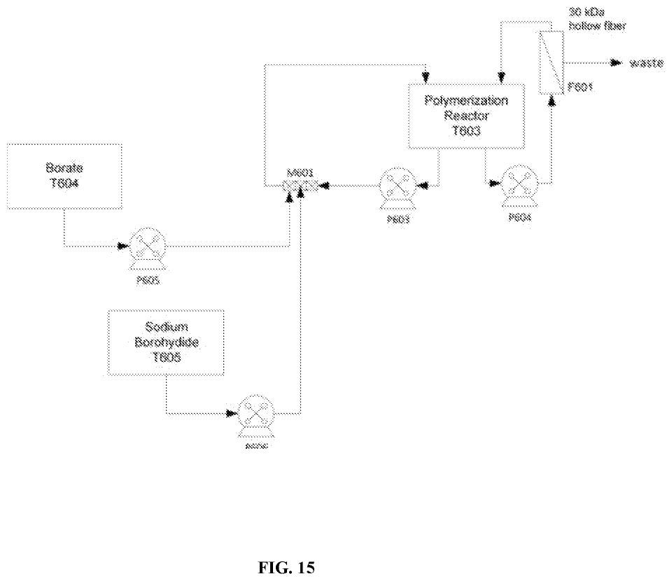

Glutaraldehyde-hemoglobin bonds are stabilized by reduction with sodium borohydride as summarized in FIG. 15. Sodium borohydride decomposes in aqueous solution at neutral pH to form molecular hydrogen and sodium borate. Diafiltration of polymerised hemoglobin with sodium borate buffer is carried out to stabilize sodium borohydride and limit hydrogen gas formation. Borate buffer is composed of 4.58 g/L sodium borate decahydrate and 0.91 g/L sodium hydroxide in Water for Injection. The buffer is filtered through a 10,000 Da membrane to reduce pyrogen content and is stored in a 20 L flexible bag (T605). The borate buffer is pumped into T603, through the recirculation loop, initially at a flow rate of 250 mL/min. Simultaneously, the polymerized hemoglobin solution is diafiltered by pumping through a 30,000 Da hollow fiber membrane at a flow rate of 1,000 mL/min. The borate addition flow rate is adjusted to equal that of the diafiltration permeate rate, approximately 250 mL/min. Diafiltration with borate buffer continues until the volume corresponding to 3 times that of the polymerized hemoglobin solution have been added.

Sodium borohydride solution is comprised of 9.45 g/L sodium borohydride, 4.58 g/L sodium borate decahydrate and 0.91 g/L sodium hydroxide in Water for Injection. The solution is filtered through a 10,000 Da membrane to reduce pyrogen content and stored in a 2 L flexible bag (T606). Sodium Borohydride solution (0.6 L) is pumped into T603, through the recirculation loop, initially at a flow rate of 7 mL/min and the temperature of T603 controlled at 20±2° C. The borohydride reaction continues for 60 minutes after all the solution has been added, with continuous recirculation of the polymerized hemoglobin solution.

The stabilized polymerised hemoglobin solution is concentrated across the 30 kDa ultrafiltration membrane (F601) to a hemoglobin concentration of 100±5 g/L. Boron containing components (sodium borate/sodium borohydride) are removed and the pH reduced to 8.0-8.4 by diafiltration of the polymerised hemoglobin across 30 kD ultrafiltration membrane (F601) with Diafiltration Solution A (6.67 g/L sodium chloride, 0.30 g/L potassium chloride, 0.20 g/L calcium chloride dihydrate, 0.445 g/L sodium hydroxide, 2.02 g/L N-acetyl-L-cysteine, 3.07 g/L sodium lactate, pH=4.9-5.1).

Examples of parts used for the polymerization process is given in TABLE 17 below, and examples of parts used for polymerization in-process testing is given in TABLE 18 below.

TABLE 17

| ID | Part | Manufacturer |
|---|---|---|
| T502 | 20 L Ready Circuit | GE Healthcare |
| T601 | 50 L Ready Circuit | GE Healthcare |
| T602 | 50 L Ready Circuit | GE Healthcare |
| T603 | 50 L Ready Circuit | GE Healthcare |
| T604 | 50 L Ready Circuit | GE Healthcare |
| T605 | 20 L Ready Circuit | GE Healthcare |
| P601 | Stainless Digital Process Pump | Masterflex |
| P602 | Stainless Digital Process Pump | Masterflex |
| P603 | Stainless Digital Process Pump | Masterflex |
| P604 | Stainless Digital Process Pump | Masterflex |
| P605 | Stainless Digital Process Pump | Masterflex |
| P606 | Stainless Digital Process Pump | Masterflex |
| M601 | Static Mixer | Kobi |
| F601 | 30,000 Da Hollow Fiber | Sartorius |

TABLE 18

| Test Material/Parameter | Measurement |
|---|---|
| Column eluate | UV280 |
| Chromatography Buffers | LAL |

### Sterile Filtration

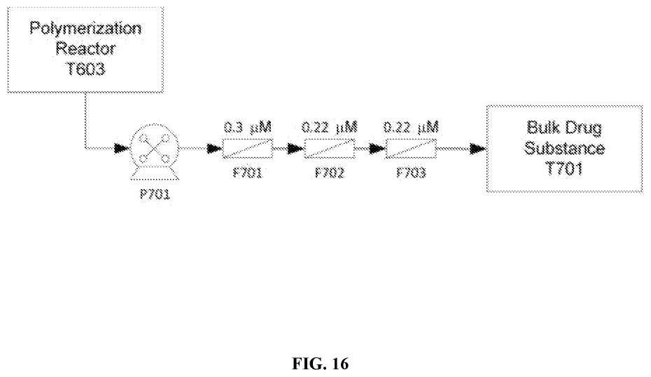

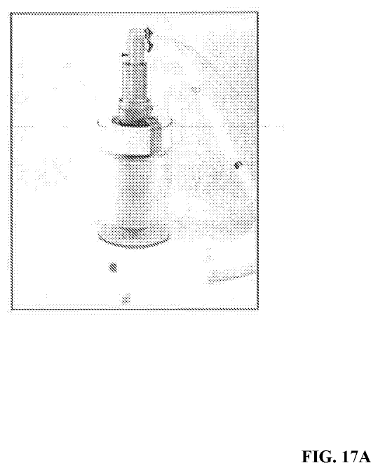

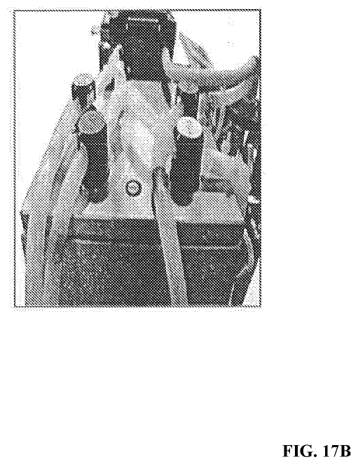

Final polymerised haemoglobin solution is filtered through a 0.5 μm depth filter (F701), a sterilizing grade 0.2 μm membrane filter (F702), and a 2ndsterilizing grade 0.2 μm membrane filter (F703), into a 20 L GE Ready Circuit flexible bag (T701). The bulk holding tank is stored under nitrogen until use. A schematic of the sterile filtration process is depicted in FIG. 16. Examples of parts used for the sterile filtration process is given in TABLE 19 below.

TABLE 19

| ID | Part | Manufacturer |
|---|---|---|
| T603 | 50 L Ready Circuit | GE Healthcare |
| T701 | 20 L Ready Circuit | GE Healthcare |
| P701 | Stainless Digital Process Pump | Masterflex |
| P602 | Stainless Digital Process Pump | Masterflex |
| F701 | 0.3 μM depth filter | Sartorius |
| F702 | 0.22 μM sterilization filter | Sartorius |
| F703 | 0.22 μM sterilization filter | Sartorius |

### Example 3: Devices and Assemblies for Manufacture and Purification Processes

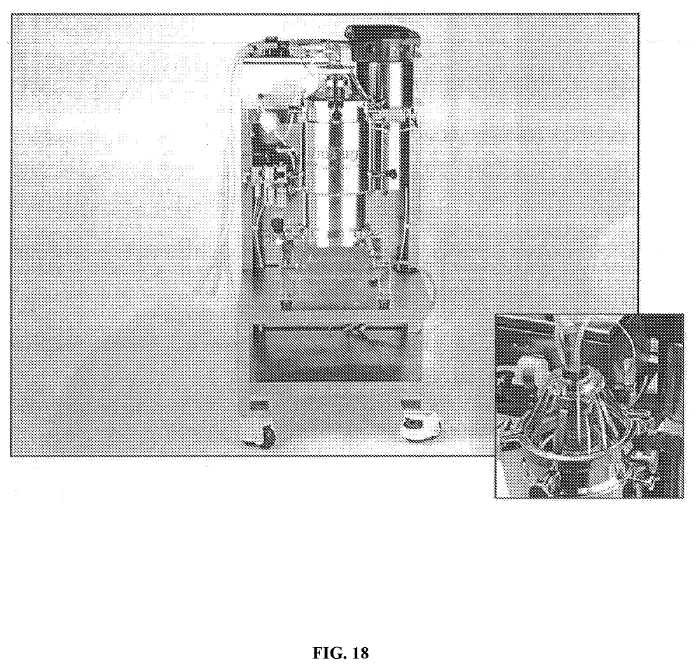

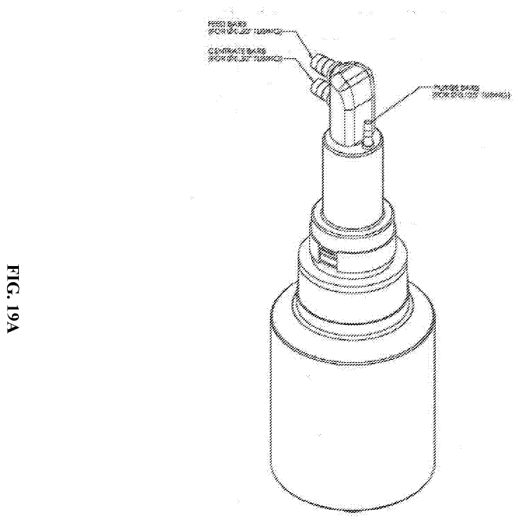

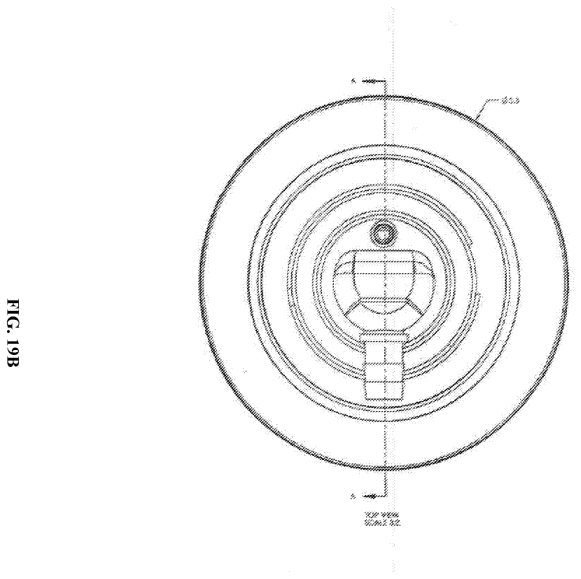

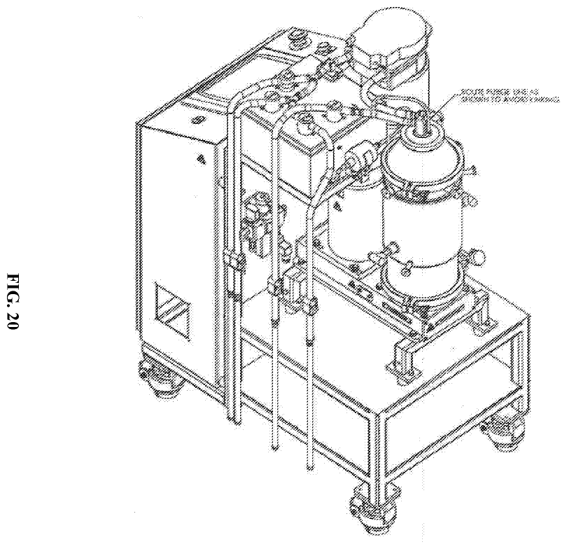

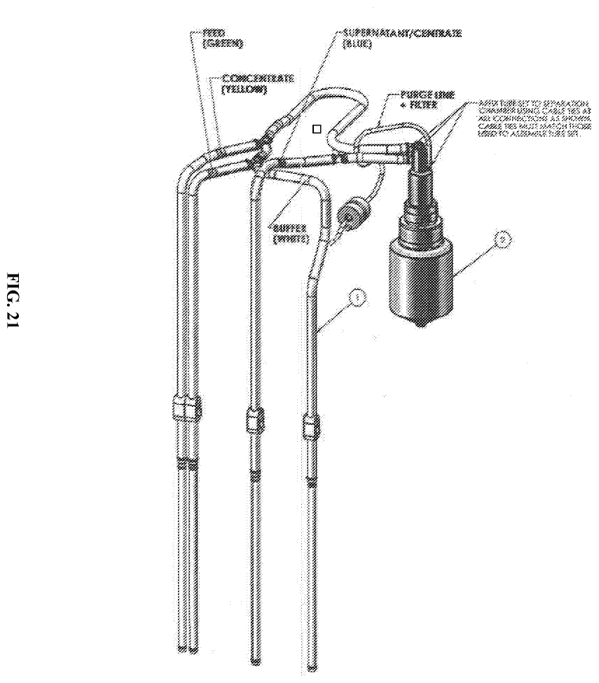

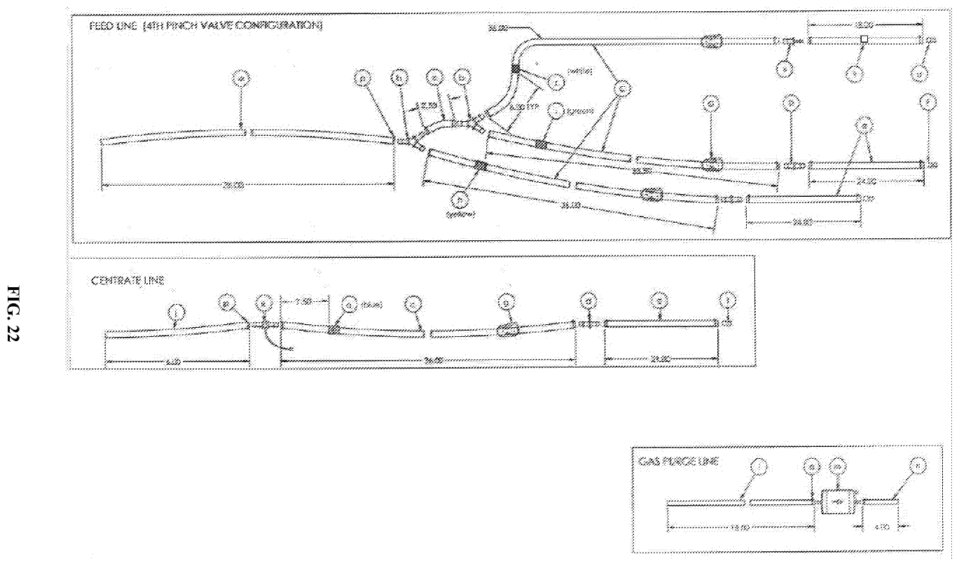

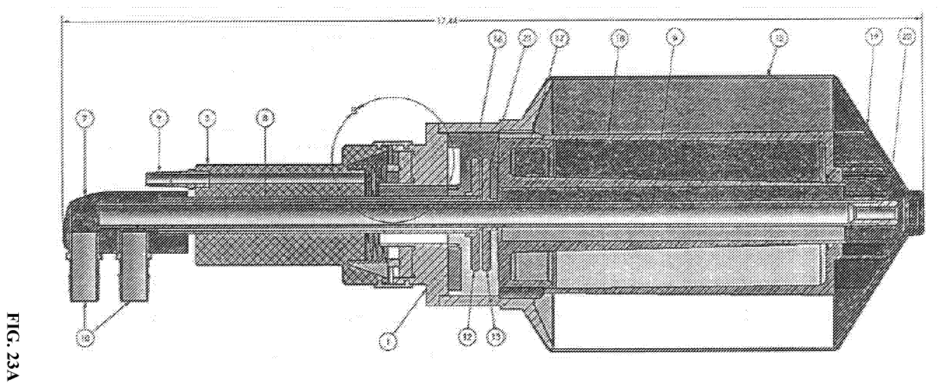

The protein (e.g. hemoglobin) purification process involves use of a separation system (FIG. 18). This separation system includes a separation chamber (FIG. 19A-FIG. 19B) and a tubeset assembly (FIG. 22) which assembles together (FIG. 21) and can be installed into a module system (FIG. 20) for extracting protein (e.g. hemoglobin) from a solution (e.g. blood). An additional device (FIG. 23) can be included in the separation system for protein purification.

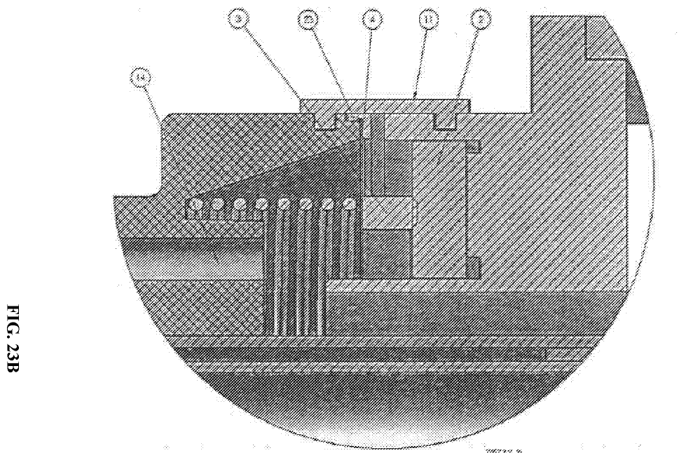

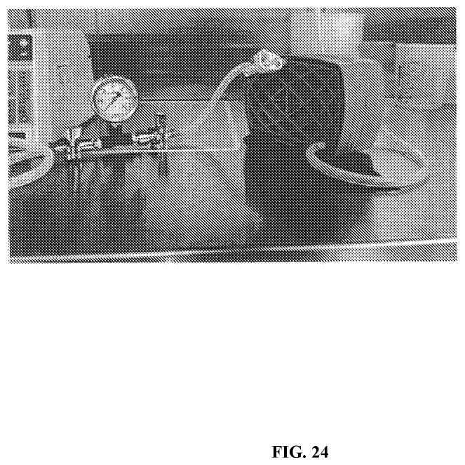

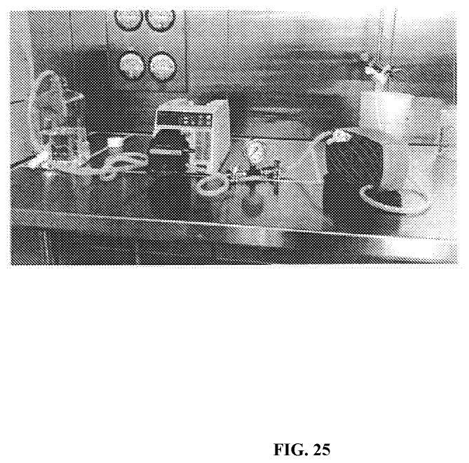

Blood depth filtration can be performed using a Millipore Clarisolve 60HX of like device (FIG. 24). The Millipore Clarisolve 60HX or like device can be connected to an assembly (FIG. 25) for blood depth filtration.

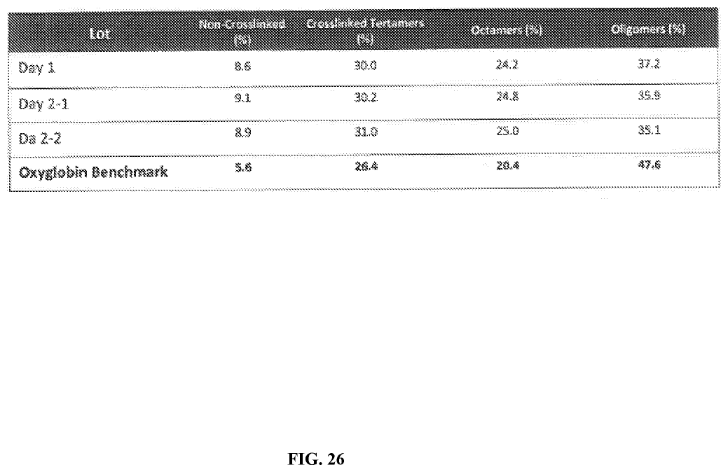

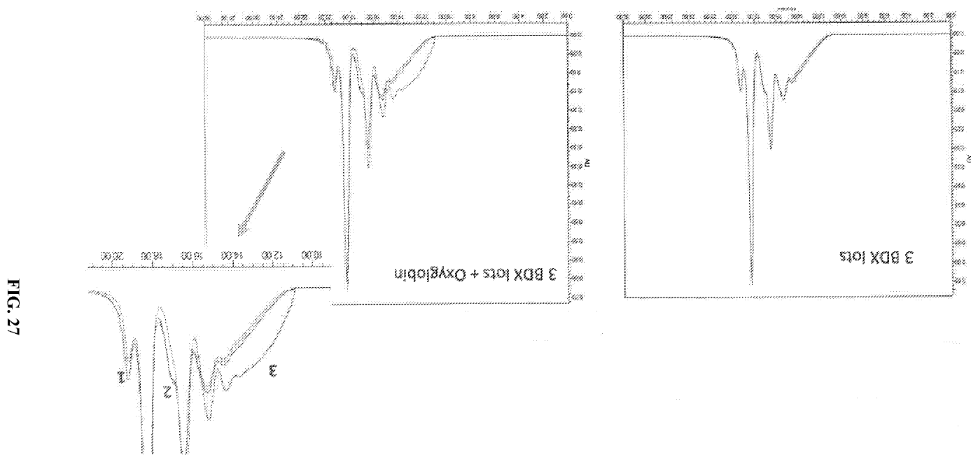

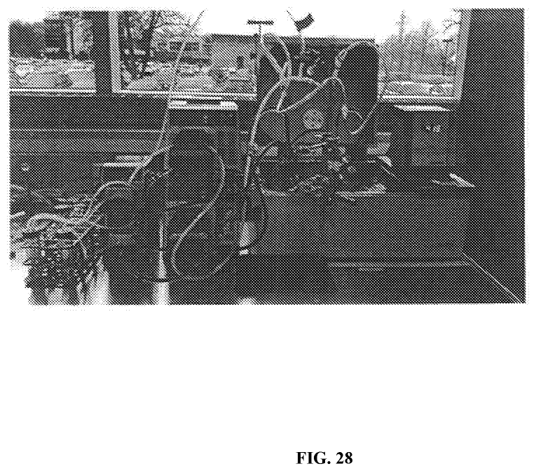

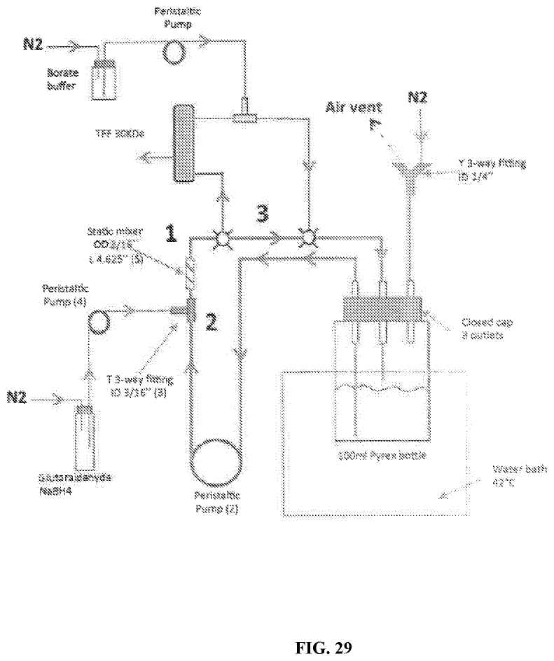

An example of a polymerization assembly is depicted as both a schematic (FIG. 29) and an image (FIG. 28). In this assembly, different glutaraldehyde/bHB proportions and types of manifold were tested. Three polymerization reactions were performed on 2 days to evaluate reproducibility with the optimized manifold. Testing parameters included 1 lot on 04 may and 2 lots on 05 May with 18 g of material per test and 29 mg gluteraldehyde per gram of hemoglobin (bHB). Testing apparatus in FIG. 28 has a static mixer 3/16″ OD×4,625 length, a T-shaped connector instead of Y-shaped to avoid Glut reflux, valves on retentate tubing for closed system conc./diaf., and continuous N2 sparging. Graphs (FIG. 27) and a chart (FIG. 26) containing protein cross-linking distribution data after polymerization processing of protein (hemoglobin) were obtained.

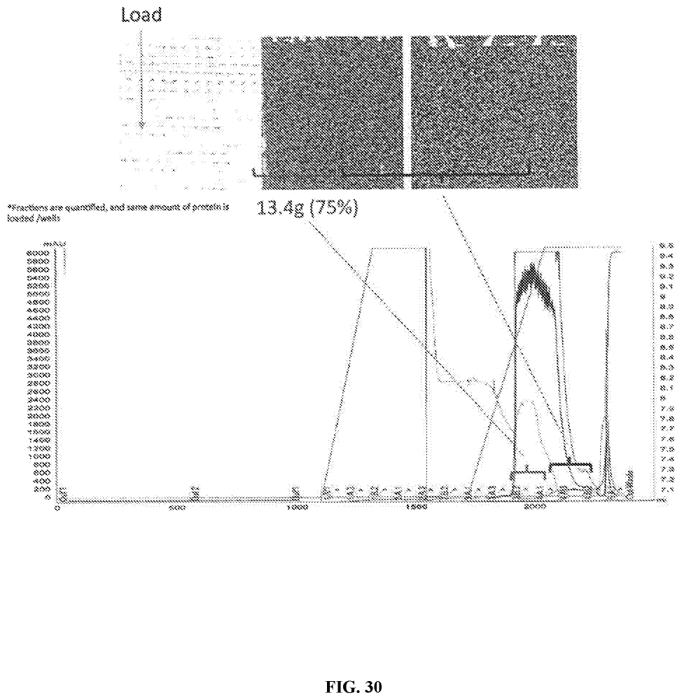

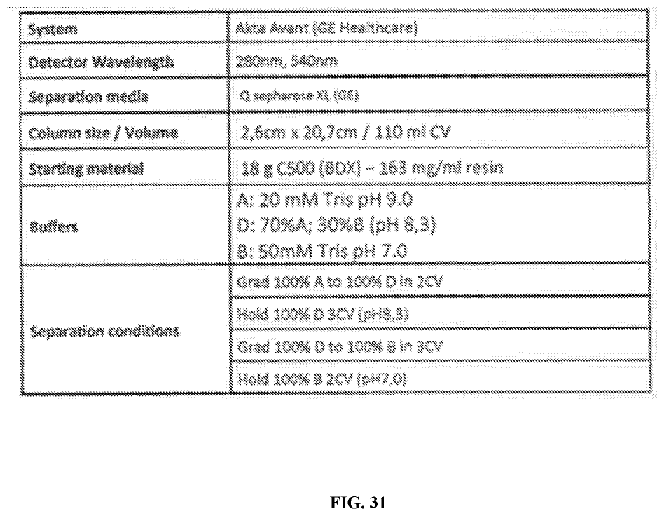

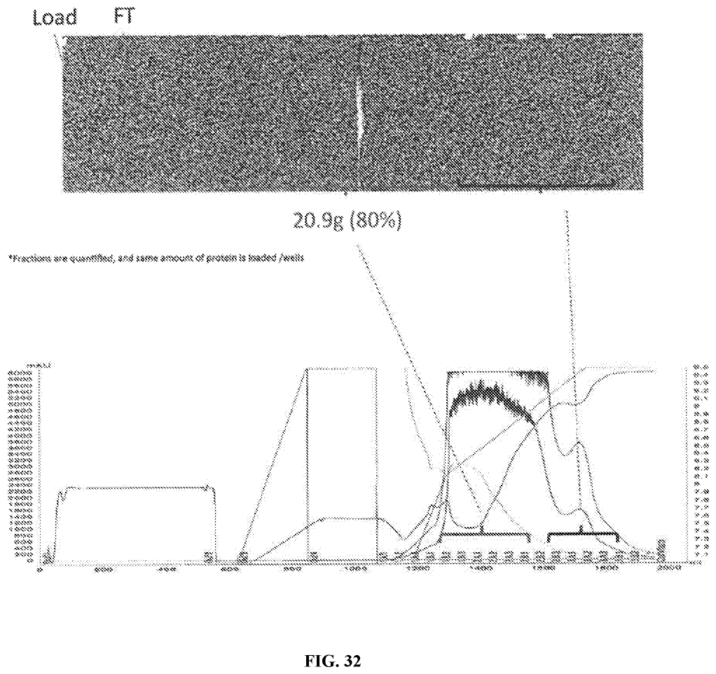

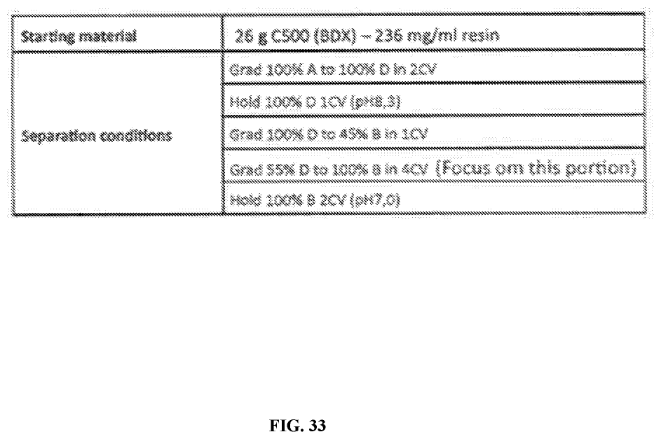

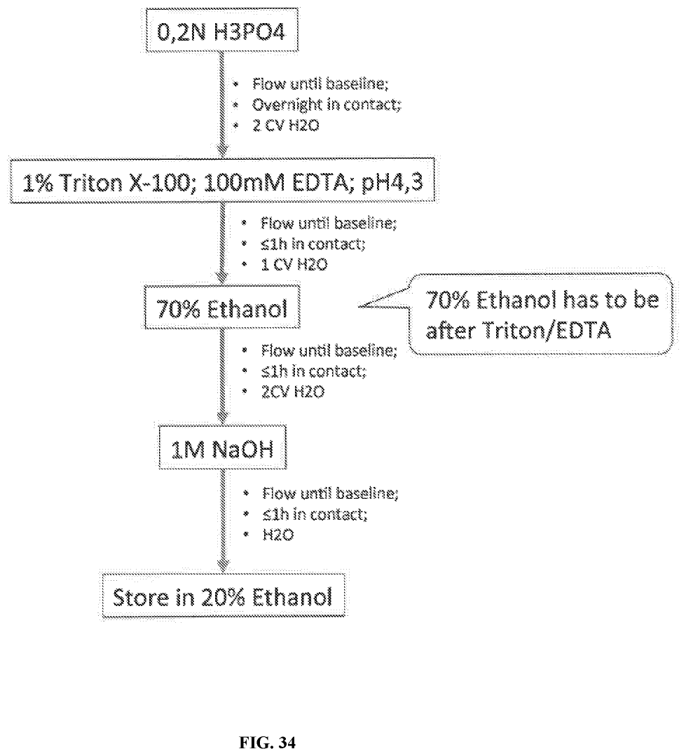

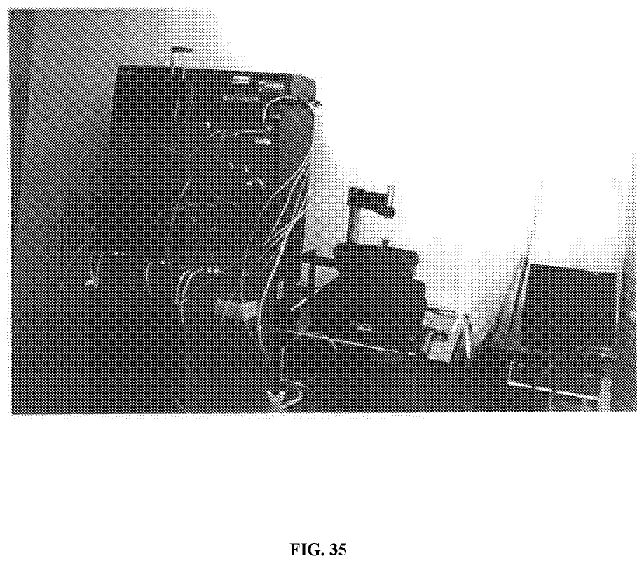

An example of a chromatography system assembly for protein purification is shown in FIG. 35. Two different gradient optimizations were performed for a C800 QEX (or equivalent) chromatography system. Graphs, images, and charts containing chromatography optimization 1 data are depicted in FIG. 30-FIG. 31. Graphs, images, and charts containing chromatography optimization 2 data are depicted in FIG. 32-FIG. 33. A flow chart for optimization of CIP of Q sepharose XL in a C800 QEX (or equivalent) chromatography system is shown in FIG. 34. In some cases of chromatography processing a 412 ml column (5 cm diameter) was loaded with 180-220 mg hemoglobin (bHB)/ml resin. Three runs were completed to process C500 1705A. A fraction collector was used for first runs and buffers were continuously N₂ sparged. The fraction collector is designed to be wrapped in an atmosbag inflated with N₂. In some instances, the gradient method was optimized on a 2.6 cm diameter column.

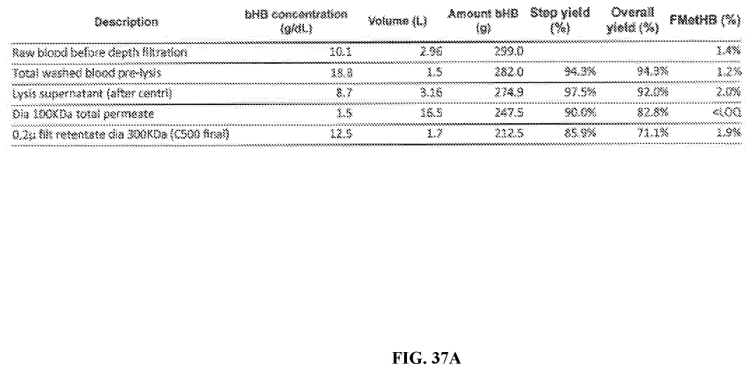

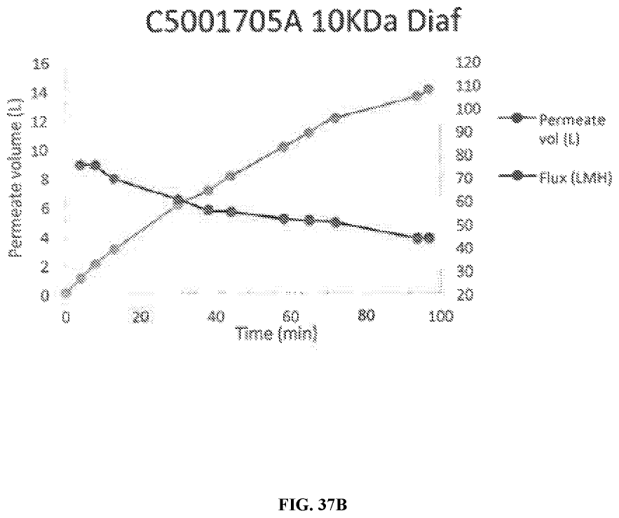

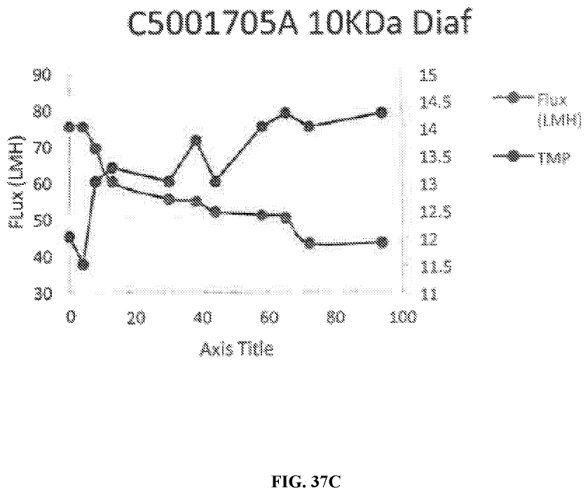

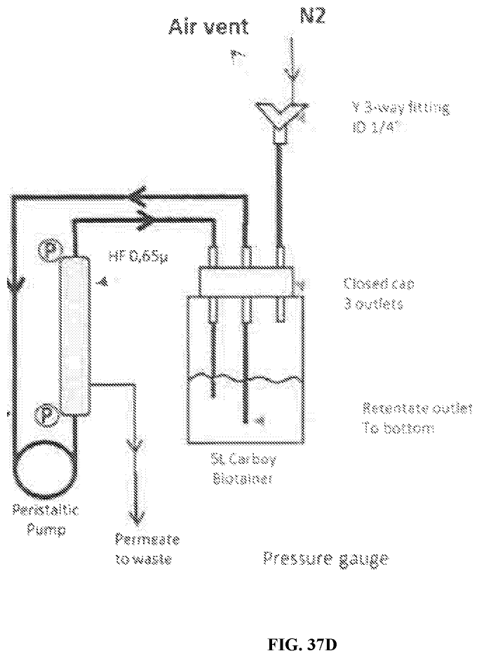

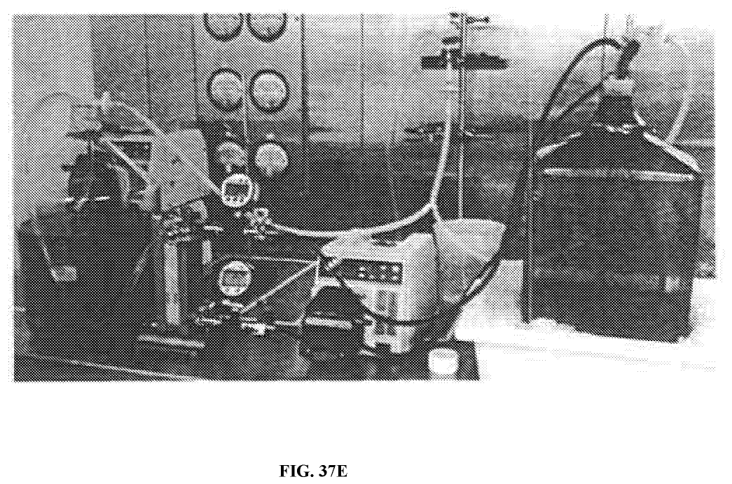

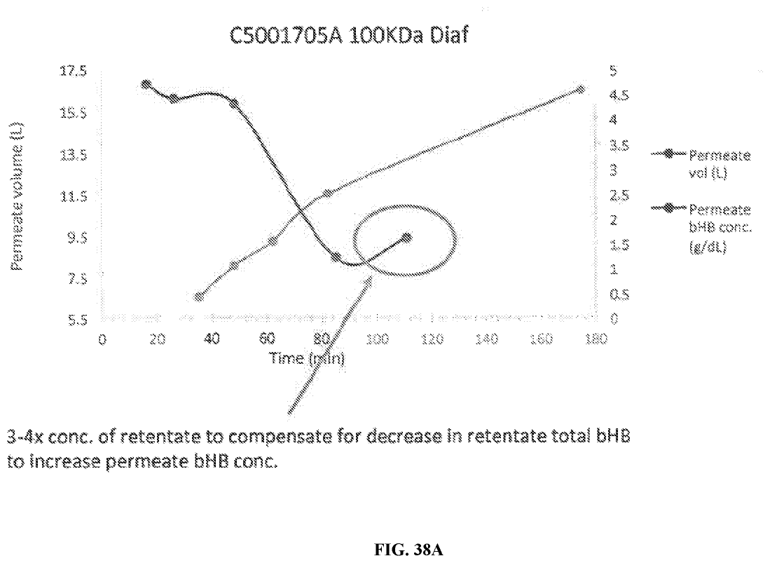

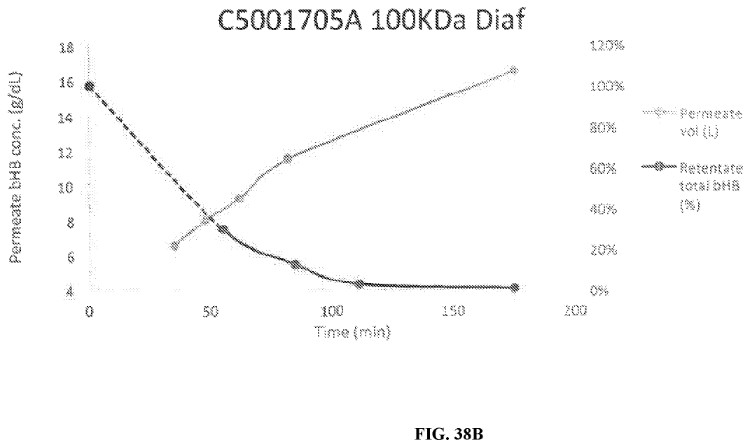

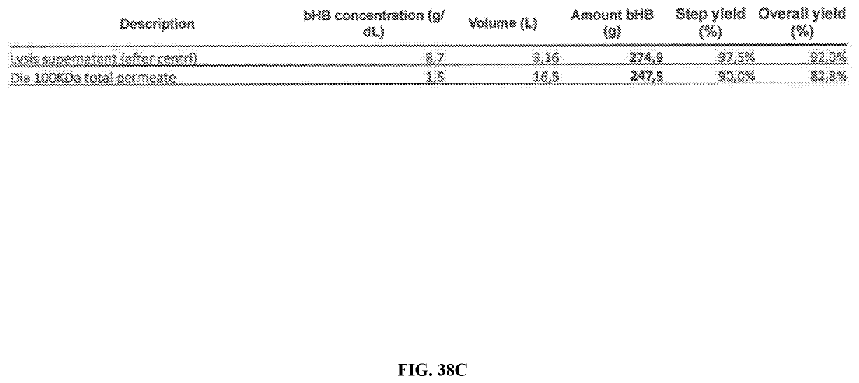

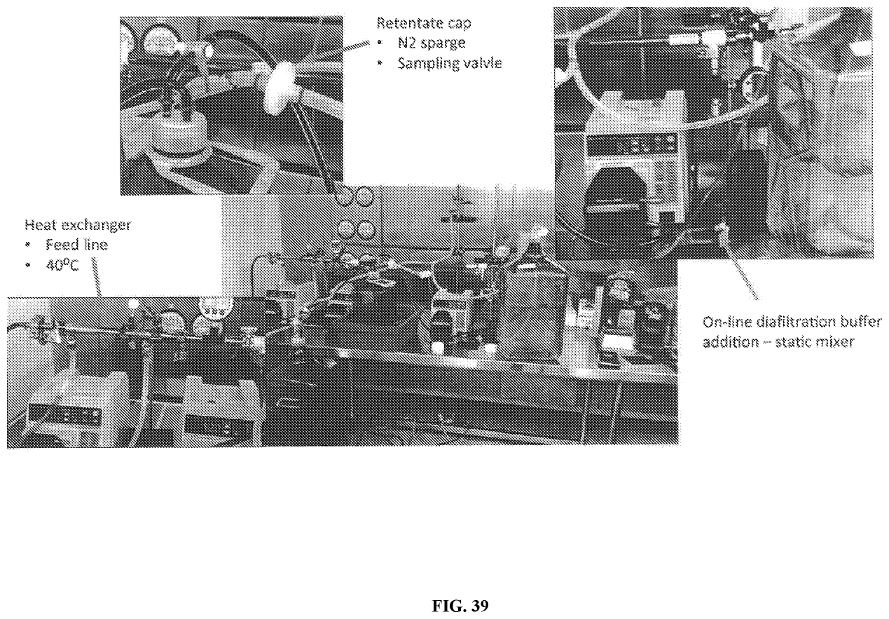

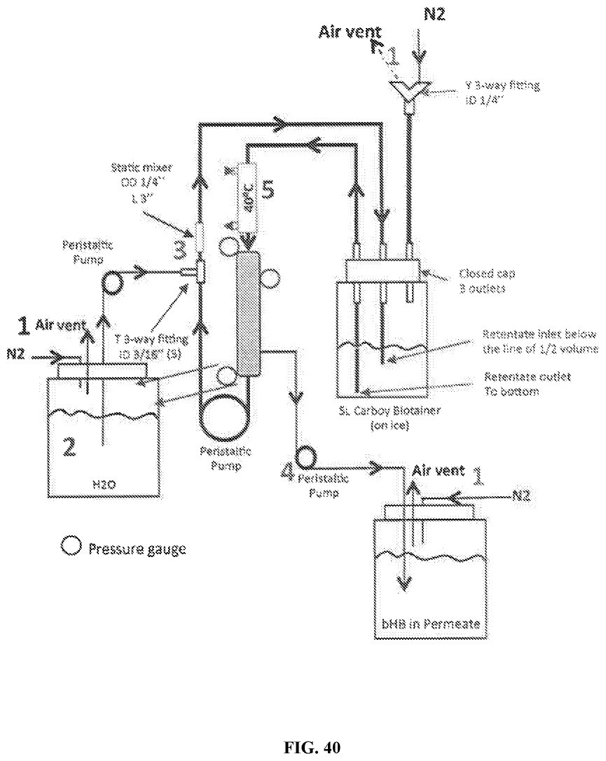

FIG. 37A-FIG. 37E depict charts, graphs, and images of a 10 KDa diafiltration process for protein purification. FIG. 38A-FIG. 38C depict a series of charts and graphs of a 100 KDa diafiltration process for protein purification. An example of an assembly for the 100 KDa diafiltration process as an image (FIG. 39) and a schematic (FIG. 40) are shown. The 100 KDa diafiltration process involves constant N₂ sparging of retentate, permeate, and diafiltration buffer (H2O); uses diafiltration H₂O (MilliQ H₂O) at <0.005EUml diafiltered with 10 KDa membrane; involves addition of diafiltration buffer through a T fitting with a static mixer directly in the retentate tube to improve the homogeneity of the retentate without using magnetic stirrer; includes permeate flow control with peristaltic pump to prevent formation of gel layer and flux reduction and to bridge with large pilot scale; and includes brief passage of the feed through 40° C. heat exchanger before entering the membrane which promotes increase in the proportion of the transient dimeric bHB form to improve diafiltration efficacy and yield.

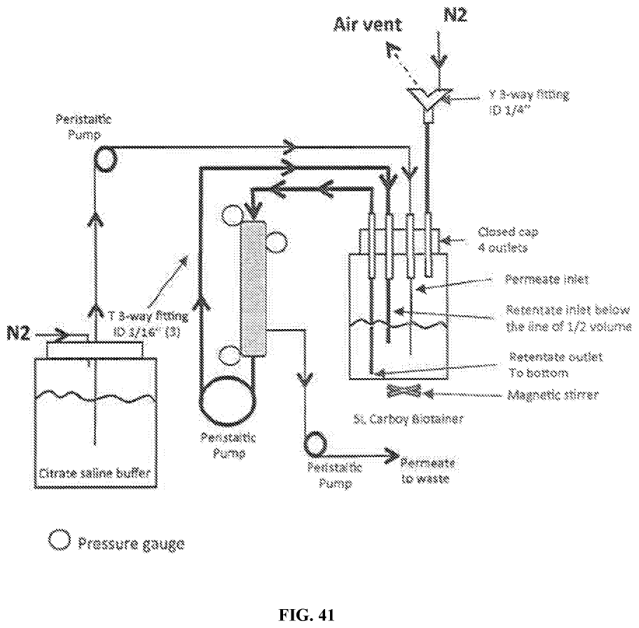

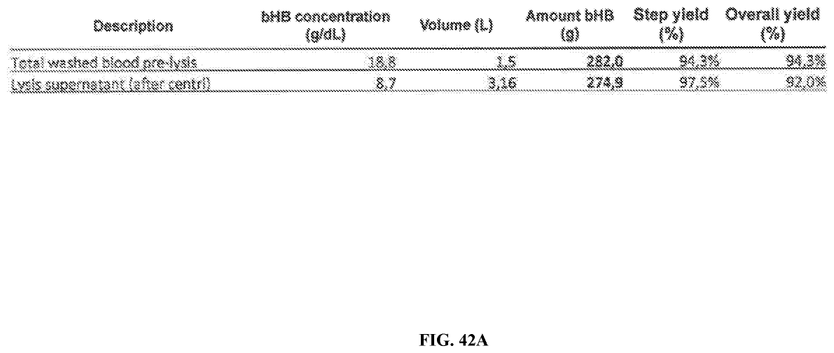

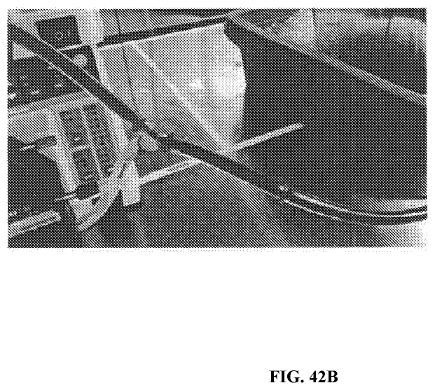

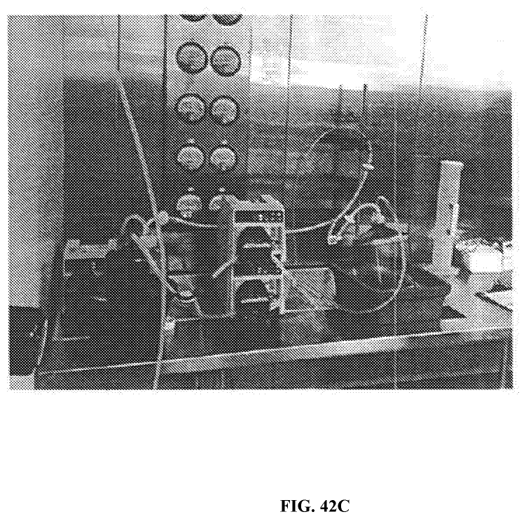

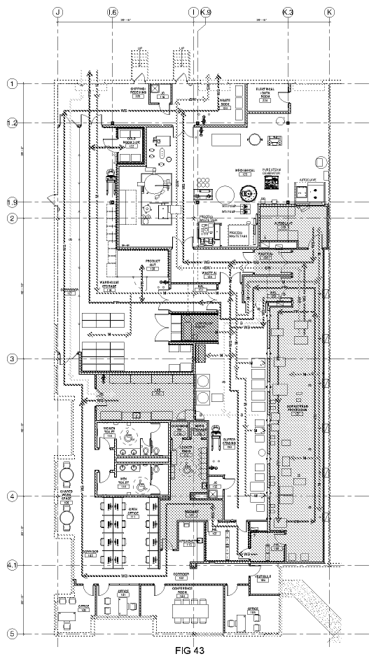

FIG. 41 depicts a schematic of a hollow fiber washing process. This process is employed on the anticoagulated blood cells before lysis. It is performed in many ways to keep the red cell intact and to ensure hemoglobin does not suffer from endotoxin and other lipid exposures. FIG. 42A-FIG. 42C are a series of images and charts depicting data from blood washing and lysis processes.

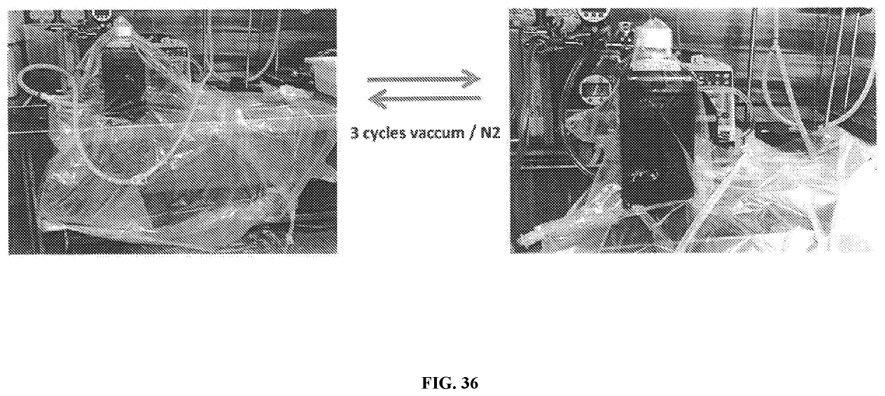

FIG. 36 is an image depicting storage of protein product C500 which can be stored at 4° C. for up to 4 weeks. This product is and intermediate material which is not chemically treated but is deoxygenated to ensure low to no oxidative activity. Sterility filtration is a benefit in the life extension to permit usable material to be drawn from the storehouse of material.

### Example 4 Modified Hemoglobin Protein Based Oxygen Carrier

Several lots of Modified Hemoglobin Protein Based Oxygen Carrier that was produced according to the disclosure were analyzed according to standard test methods. The results of lots are depicted in tables 20-23 below.

TABLE 20
Certificate Test Date: 25 Jun. 2018

| | Approved Test | | | |
|---|---|---|---|---|
| Release Tests | Methods | Unit | Specification | Test Result |
| 1. Potency | | | | |
| Total Hb | Co-oximetry | g/dL | 5.5-7.5 | 5.5 |
| Met Hb | Co-oximetry | % | <10 | 2.0 |
| Oxy Hb | Co-oximetry | % | <10 | 2.0 |
| 2. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.022 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 3.67 |
| 3. Identity | | | | |
| Appearance | Visual | N/A | Deep Purple | Deep Purple |
| pH | Potentiometry | N/A | 7.6-7.9 @18-22° C. | 7.74 |
| Ion Concentration | | | | |
| Na⁺ | Ion selective electrode | mM | 145-160 | 160 |
| K⁺ | Ion selective electrode | mM | 3.5-5.5 | 3.9 |
| Cl⁻ | Ion selective electrode | mM | 105-120 | Not Tested |
| Ca²⁺ | Ion selective electrode | mM | 0.5-1.5 | 0.74 |

TABLE 21
Certificate Test Date: 2 Jul. 2018

| | Approved Test | | | |
|---|---|---|---|---|
| Release Tests | Methods | Unit | Specification | Test Result |
| 4. Potency | | | | |
| Total Hb | Co-oximetry | g/dL | 5.5-7.5 | 6.3 |
| Met Hb | Co-oximetry | % | <10 | 2.2 |
| Oxy Hb | Co-oximetry | % | <10 | 2.1 |
| 5. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.054 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 4.49 |
| 6. Identity | | | | |
| Appearance | Visual | N/A | Deep Purple | Deep Purple |
| pH | Potentiometry | N/A | 7.6-7.9 @18-22° C. | 7.71 |
| Ion Concentration | | | | |
| Na⁺ | Ion selective electrode | mM | 145-160 | 154 |
| K⁺ | Ion selective electrode | mM | 3.5-5.5 | 3.7 |
| Cl⁻ | Ion selective electrode | mM | 105-120 | Not Tested |
| Ca²⁺ | Ion selective electrode | mM | 0.5-1.5 | 0.71 |

TABLE 22
Certificate Test Date: 16 Jul. 2018

| | Approved Test | | | |
|---|---|---|---|---|
| Release Tests | Methods | Unit | Specification | Test Result |
| 7. Potency | | | | |
| Total Hb | Co-oximetry | g/dL | 5.5-7.5 | 6.7 |
| Met Hb | Co-oximetry | % | <10 | 3.1 |
| Oxy Hb | Co-oximetry | % | <10 | 3.0 |
| 8. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.044 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 5.95 |
| 9. Identity | | | | |
| Appearance | Visual | N/A | Deep Purple | Deep Purple |
| pH | Potentiometry | N/A | 7.6-7.9 @18-22° C. | 7.67 |
| Ion Concentration | | | | |
| Na⁺ | Ion selective electrode | mM | 145-160 | 154 |
| K⁺ | Ion selective electrode | mM | 3.5-5.5 | 3.8 |
| Cl⁻ | Ion selective electrode | mM | 105-120 | Not Tested |
| Ca²⁺ | Ion selective electrode | mM | 0.5-1.5 | 0.74 |

TABLE 23
Certificate Test Date: 23 Jul. 2018

| | Approved Test | | | |
|---|---|---|---|---|
| Release Tests | Methods | Unit | Specification | Test Result |
| 10. Potency | | | | |
| Total Hb | Co-oximetry | g/dL | 5.5-7.5 | 7.0 |
| Met Hb | Co-oximetry | % | <10 | 1.2 |
| Oxy Hb | Co-oximetry | % | <10 | 1.9 |
| 11. Purity | | | | |
| Sterility | Sterility test | N/A | Pass | Pass |
| Endotoxin Level | Kinetic turbidimetric | EU/mL | <0.05 | <0.04 |
| Glutaraldehyde | HPLC | ug/mL | <0.15 | 0.038 |
| N-acetyl-cysteine | HPLC | % | <0.24 | Not Tested |
| Molecular Weight | | | | |
| Distribution | | | | |
| MW >500,000 | HPLC-SEC | % | <15 | Not Tested |
| MW <32,000 | HPLC-SEC | % | <5 | 5.36 |
| 12. Identity | | | | |
| Appearance | Visual | N/A | Deep Purple | Deep Purple |
| pH | Potentiometry | N/A | 7.6-7.9 @18-22° C. | 7.72 |
| Ion Concentration | | | | |
| Na⁺ | Ion selective electrode | mM | 145-160 | 155 |
| K⁺ | Ion selective electrode | mM | 3.5-5.5 | 3.8 |
| Cl⁻ | Ion selective electrode | mM | 105-120 | Not Tested |
| Ca²⁺ | Ion selective electrode | mM | 0.5-1.5 | 0.71 |

### Example 5. cGMP Manufacture Modified Hemoglobin Protein Based Oxygen Carrier

Referring to FIG. 43, a commercial scale manufacturing facility is depicted. The main manufacturing suite room 127 is designed to meet Grade C/ISO8 specifications. This room is the main processing room where the hemoglobin solution(s) (i.e. raw material diluted with water) will be further purified by dedicated ion exchange chromatography according to the disclosure. The eluate is collected in an appropriate vessel so as to limit and prevent oxygen and particulate exposure. Handling and connecting are performed via tubing welders and appropriate closed containers thus mitigating all risk of room environmental exposure. Materials are them concentrated across a 30 kD TFF membrane. A bolus of NaCl buffered solution is added to the highly purified hemoglobin solution to allow for deoxygenation across a hydrophobic gas exchange membrane.

The hemoglobin solution is, filtered into the storage buffer containing an oxygen scavenger and concentrated to achieve the target hemoglobin concentration. The hemoglobin solution is then “0.2 micron filtered” into a pre-sterilized bag for storage until further processing (no open system transfers). This room also contains the process equipment for polymerizing the hemoglobin, quenching the reaction and exchanging the buffers using 30 kD membranes. Each vessel in the polymerization system also recirculates through a closed system hydrophobic gas exchange membranes to remove any oxygen introduced to the system by the addition of chemical and buffers to the process. The final polymerized hemoglobin product will be “0.22 micron filtered” into a pre-sterilized vessel. The final product will be stored in the warehouse in a secure area until release whereby it will be shipped to the contract filling facility.

In further reference to FIG. 43, the manufacturing support suite room 130 is designed to meet Grade D/ISO9 specifications. This room will support the main processing area by formulating buffers used in the process. The chemicals used in the buffer formulation will be weighed in a containment hood to control particles. The buffers will be supplied to the process with tubing passed through ports in the walls and sealed with iris valves. These ports will also be used to transfer process waste fluids to a waste transfer header with will flow to a waste accumulation tank below grade.

In compliance with pharmaceutical defined SOPs, the room cleaning will be performed each working day with a quaternary ammonium “sanitant” according to the defined SOP. Monthly the rooms will be cleaned with a sporicidal agent or in response to excursions in the environmental monitoring program. The process will be performed through the use of closed pre-sterilized single-use systems. Sampling will be performed on vessels that have been tubing welded onto the system to maintain the closed system status.

As depicted in FIG. 43, the component prep room 128 is designed to meet Grade C specifications. The room will be used to prepare assemblies to use in the process of sterilization. The room includes USP purified water for rinsing materials and WFI for performing final rinse of components as needed. The room will also include an integrity tester for the pre and post-use integrity testing to be performed.

Also as shown in FIG. 43, the utility room 123 contains utilities to support the facility functions. This includes a plant steam boiler, air compressor, nitrogen/argon system, vacuum system, USP water system, pure steam generator with WFI condenser, WFI system, and the wastewater neutralization system. The mechanical side of the autoclave is also accessed from this space. The waste neutralization system will be the batch discharge type to ensure compliance with the pH discharge limits and to provide good flow for accurate measurement.

As depicted in FIG. 43, the warehouse room 119 is used to securely store the materials used in the production process which includes an addition secured are for final bulk product storage (room 120) and a cold room (room 122) for storage of the incoming hemoglobin solution. Incoming chemicals will be purchased with representative samples for QC testing.

The quality control lab room 118 will be used for the testing sample to support the ongoing operations. The bulk of the testing will be contracted out to a yet to be identified appropriate contract testing lab.

### Raw Material Source

The starting material for the process is bulk bovine hemoglobin which has been collected from a controlled donor herd. The collected red cells are washed either by diafiltration across a tangential flow filtration system or by centrifugation in a single-use disposable centrifuge. The red cells are then lysed by osmotic pressure then the hemoglobin is filtered across a 100 kD TFF membrane. The permeate is collected and concentrated across a 30 kD TFF membrane. Once the hemoglobin is at the target concentration, the hemoglobin solution is “0.22 micron filtered” into bags and stored at 2-8° C.

### Country of Origin

All animals are of US origin. The US is a GBR level II country as defined in the European Union document “Update of the Opinion of the Scientific Steering Committee on the Geographical Risk of Bovine Spongiform Encephalopathy (GBR), Adopted on 11 Jan. 2002. GBR level II indicates “it is unlikely that domestic cattle in this country are infected with the BSE-agent, but it cannot be excluded.”

### Procedures for Avoiding the Risk of Cross Contamination

Whole bovine blood for processing is collected in a dedicated collection room that is separate from the remaining processing areas of the collection room or alternatively at an abattoir in controlled space. Animals from approved suppliers enter the blood collection area from the barn. All animals, from which there is any collection, will have complete documentation according to the herd management program including origin and feed status. Following bleeding or exsanguination, the animal is removed from the blood collection room for further processing back to the herd management area or in the abattoir facility.

### Isolation of Animals

Individually identified cattle arriving at the collection station or the abattoir are controlled from managed herds. In the first instance according to a standard herd management program they will be controlled as a lot before entering the dedicated blood collection area. Cattle enter through a chute which channels them directly to the collection area or a stunning platform in the case of the abattoir. The blood collection facility is separate from the primary exsanguination (if an abattoir) or collection facility at the designated facility.

### Blood Collection

Supporting documentation and identification for each animal is verified for accuracy and completeness before each collection, and the animal is inspected for any sign of disease. Blood collection is performed using a closed system. The animal (if exsanguinated) may be immobilized and if one time harvest a non-pneumatic captive bolt method maybe used for stunning. Collection at an abattoir has never used, nor will ever use, the procedure referred to as “pithing”. Immediately after stunning if at an abattoir, chain shackles are placed around a rear hoof and the animal is hoisted to a head-down position. An overhead conveyor system moves the animal carcass along the line to the collection platform. If abattoir donation, an incision in the hide is made from the angle of the jaw to the thoracic inlet; the hide is then retracted from the exposed jugular furrow by an elastic cord wrapped around the back side of the neck.

Blood is collected in a closed manner using a stainless steel trocar inserted into the jugular vein close to the vena cava. Sanitized tubing connects the sanitized trocar to a sanitized stainless steel vessel or plastic bag, which has been prepared with sodium citrate anticoagulant. Approximately 10 to 15 liters of blood is collected in a period of approximately 30-60 seconds. After the blood is collected, the trocar is removed, and the vessel is sealed. The carcass then moves out of the dedicated Oversight Collection Facility and then onto the main abattoir processing floor and cannot be returned. If at the animal management facility where animals are bleed for a controlled volume of 2 to 5 liters, animals will be restrained during donation with the blood being collected in a sterile anticoagulant charged collection bag.

Each collection vessel holds the blood of a single animal. The unique number of each collection vessel is recorded and correlated with the animal number from a unique animal ear tag. The ear tag number is further correlated with a unique abattoir animal number used to trace the cattle through the packing plant. Animals are subsequently inspected by USDA trained inspectors for evidence of disease or contamination. The inspectors are supervised by USDA trained veterinarians. If an animal is retained by the USDA staff for further examination for any reason, the blood from that animal is discarded at the abattoir. The filled collection vessels may leave the facility, and are placed in ice and loaded onto a truck for transport to the Separation Facility. If the managed donor herd, similar cataloguing is performed and bags will be collected and cooled to be transported to initial processing facilities.

### Potential for Other Tissues to Contaminate Collected Blood

The potential for contamination by other tissues is minimal because of the closed method of blood collection and through the use of well-trained operators for the controlled and documented procedure. In the abattoir the trachea and esophagus are avoided by positioning the blade of the trocar toward the blood vessel.

The site on the skull where the animal is stunned is physically distant from the location of trocar insertion (1 meter). Because of the position in which the animal is suspended during blood collection, any fluid or bone chips from the stunning site cannot come into contact with the collection site. The collected blood does not come into contact with brain, spinal cord, eye, ileum, lymph nodes, proximal colon, spleen, tonsil, dura mater, pineal gland, placenta, cerebrospinal fluid, pituitary, adrenal, distal colon, nasal mucosa, peripheral nerves, bone marrow, liver, lung or pancreas. In addition, any potential contaminating tissue would be removed during the blood pooling process at the manufacturing plant, in which the blood is sequentially filtered by an 800μ screen, 50μ strainer and a 60μ depth filter. The 60μ depth filter has a wide distribution of pore sizes; the largest pore size is 60μ or microns.

### Water Systems

The water for injection is produced by condensing pure steam into a 2000 L storage tank maintained above 65° C. which is recirculated through a spray ball to flush all interior surfaces during operation. The hot loop does not have any direct use point but supplies a cold loop which recirculates through a heat exchanger to reduce the temperature to 25° C. One use point is at buffer preparation, and the other is in component prep to perform a final rinse before sterilization in the autoclave. The cold loop is hot water sanitized nightly for a defined time period.

The raw materials are stored at controlled room temperature except for the purified hemoglobin solution which is stored at 2 to 8° C. Standard single-use disposable product contact materials such as polypropylene, polycarbonate, silicone tubing, C-flex tubing, and bags with an inert inner layer made of ultra-low density polyethylene or equivalent are used for storage. The systems will be flushed before use to remove particulates and test for leaks before processing. If sanitation is required, the system is flushed with 0.5 M NaOH for a defined time frame then the NaOH is flushed out of the system and ensure the residual is neutralized before processing. The final product is stored at controlled room temperature.

### HVAC and Air Handling

The HV AC system provides HEPA filtered air to the clean rooms that have been cooled to reduce the moisture to less than 60% relative humidity and reheated to the desired temperature for operator comfort. The system is designed with sufficient air change rates appropriate for the classification with a pressure cascade of 0.05″ was between rooms of different classification with the main processing area at the highest pressure. The processing suite is designed with airlocks to allow the transition of people and materials to be performed with minimal impact on the processing areas. The rooms are cleaned with an approved sanitant according to a standard operating procedure. Environmental monitoring for viable and non-viable particulates will be performed on a periodic basis according to the room classification. Surface monitoring will also be performed in defined locations defined by a standard operating procedure.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for manufacturing endotoxin-free hemoglobin based drug substance comprising:
collecting bovine blood using a sterile polymeric bag contain CPD anticoagulant;
washing the collected blood by diafilitration;
lysing said bovine red blood cells producing a hemoglobin solution;
stabilizing said hemoglobin solution by removing oxygen producing deoxygenated hemoglobin solution;
filtering said deoxygenated hemoglobin solution;
purifying said deoxyenated hemoglobin solution thereby reducing non-specific blood cell components, wherein said purification is achieved via chromatography producing a purified hemoglobin solution;
stabilizing said purified hemoglobin solution by deoxygenating by filtration through about an 30,000 Da hollow-fiber membrane achieving a desired hemoglobin concentration, wherein the purified hemoglobin is deoxygenated by passage through multiple liquicell membranes;
filtering said deoxygenated purified hemoglobin solution by diafiltering against storage buffer by pumping through a 30,000 Da hollow-fiber membrane;
polymerizing said purified deoxygenated hemoglobin by cross-linking with glutaraldehyde;
stablizing said polymerized purified deoxygnated hemoglobin d reduction with sodium borohydride, wherein said stabilized polymerized purified deoxygenated hemoglobin via diafiltration of said polymerized hemoglobin producing a final polymerized hemoglobin solution; and
filtering said final polymerized hemoglobin solution.

2. The method according to claim 1 wherein said final polymerized hemoglobin solution is filtered through a 0.5 μm depth filter, a sterilizing grade 0.2 μm membrane filter, and at least one additional 2nd sterilizing grade 0.2 μm membrane filter.

3. The method according to claim 1, wherein said lysing of bovine red blood cells is by a rapid decrease in osmotic pressure resulting in cell lysis and sequential diafiltration across 100 kDa and 30 kDa membranes.

4. The method according to claim 1 wherein the step of deoxygenating said hemoglobin solution further comprises the step of pumping the hemoglobin solution through two Liquicell Membranes aligned in series at a flow rate of 500 ml-min⁻¹, with a counter-current flow of nitrogen at 75 psi until the dissolved oxygen reading is below 0.02 mg-mL⁻¹.

5. The method according to claim 1 wherein said chromatography system uses a GE Akta Biopilot chromatography system equipped with a GE Healthcare XK borosilicate column (5 cm i.d.×100 cm length) packed with Q Sepharose Fast Flow (GE Healthcare) to a bed height of 70±5 cm.

6. The method according to claim 5 wherein said chromatography system's buffers are prepared using Water for Injection and filtered through a 10 kDa membrane to further reduce pyrogen content said buffers are selected from the group consisting of (1) Buffer A; 2.42 g-L-1 tris base adjusted to pH 9.0±0.1 with acetic acid, (2) Buffer B; 6.05 g-L-1 Tris base adjusted to pH 7.0±0.1 with acetic acid and (3) Buffer C; 2.42 g-L-1 Tris base and 58.38 g-L-1 NaCl adjusted to pH 8.9±0.1 with acetic acid.

7. The method according to claim 1 wherein the hemoglobin solution is polymerized by raising the solution to 42±2° C. and a Glutaraldehyde solution is prepared at a concentration of 6.2 g/L in a temperature controlled Wave bag (T602) and heated to 42±2 C and said Glutaraldehyde solution is pumped into T603 at a rate of 10 mL/min until the ratio of glutaraldehyde to hemoglobin is approximately 0.029:1.

8. The method according to claim 7 wherein the glutaraldehyde is added through a static mixer in a recirculation loop to ensure rapid and homogeneous mixing with the hemoglobin and the temperature of the reaction mixture is cooled to 22±2° C.

9. The method according to claim 8 where the reaction mixture is concentrated by diafiltration through a 30,000 Da hollow-fiber membrane (F601) to a hemoglobin concentration of 80±5 g/L.

10. The method according to claim 1 wherein said sodium borohydride solution is comprised of 9.45 g/L sodium borohydride, 4.58 g/L sodium borate decahydrate and 0.91 g/L sodium hydroxide in Water for Injection and said sodium borohydride solution is filtered through a 10,000 Da membrane to reduce pyrogen content.
